(19) United States
(12) Patent Application Publication
(10) Pub. No.: US 2025/0268860 A1
(43) Pub. Date: Aug. 28, 2025

(54) COMPOSITION FOR USE IN THE CUTANEOUS SYNTHESIS OF HYALURONIC ACID, IN AN IMMUNO-CUTANEOUS RESPONSE AND TO IMPROVE SKIN

(71) Applicant: JR, PARIS (FR)

(72) Inventor: Philippe BERNARD, LA FERTE SAINT-AUBIN (FR)

(21) Appl. No.: 18/703,010

(22) PCT Filed: Jun. 21, 2022
(86) PCT No.: PCT/FR2022/051204

§ 371 Date: Apr. 19, 2024

(30) Foreign Application Priority Data
Oct. 22, 2021 (FR) 2111237

(51) Int. Cl. A61K 31/352; A61K 8/37; A61K 8/49; A61K 8/73; A61K 31/216; A61K 47/36; A61P 17/00; A61P 37/06; A61Q 19/00; A61Q 19/08

(52) U.S. Cl. CPC A61K 31/352; A61K 8/375; A61K 8/498; A61K 8/73; A61K 31/216; A61K 47/36; A61P 17/00; A61P 37/06; A61Q 19/007; A61Q 19/08

(57) ABSTRACT

A composition comprising baicalin or a derivative thereof, more particularly in combination with chlorogenic acid or a derivative thereof, as well as the cosmetic use of such a composition and its medical applications.

The invention relates to a composition comprising baicalin or a derivative thereof optionally in combination with chlorogenic acid or a derivative thereof, in particular for the use of this composition in the cutaneous synthesis of hyaluronic acid, for its use in the immunocutaneous response and for improving the general appearance of the skin.

The invention also relates to a composition according to the present invention for use in the cellular synthesis of beta-defensin, and also to the treatment of disorders involving cellular synthesis of beta-defensin.

Hyaluronic acid (HA) is a glycosaminoglycan, a sulfate-free amino polyoside composed of repeating units of D-glucuronic acid and N-acetyl-D-glucosamine, which is capable of binding water. Thus, glycosaminoglycans associated with a bearing protein form proteoglycans. These proteoglycans take up water molecules and constitute a gel, a water reservoir for the skin. The skin's HA content is therefore important for maintaining good skin hydration. In normal skin, HA is synthesized mainly by dermal fibroblasts and epidermal keratinocytes. By virtue of its residues bearing a negative charge, HA acts as a water pump, allowing the skin's elasticity to be maintained. HA plays a key role in controlling the diffusion of foods, hormones, vitamins and inorganic salts from connective tissue, and in cleaning up metabolic waste products that may induce inflammatory reactions.

Factors such as the sun, stress, pollution and age lead to a loss of HA from the skin, reducing the amount of water retained in the connective tissue. The skin then undergoes an aging process, leading to an increase in fibrosis and a decrease in elastic fiber content. In normal human skin, HA exists as a high molecular weight polymer (600 000-1 000 000 Da). Physiological degradation of HA in the skin involves (i) internalization by keratinocytes and (ii) intracellular fragmentation into fragments of intermediate size by hyaluronidases (60 000-300 000 Da). The fragmented HA is released by the keratinocytes, crosses the basement membrane and is released directly into the lymphatic vessels. Thus, loss of HA from the skin may, in certain extreme cases, cause certain skin disorders or even pathologies leading to dry skin, such as atopy, psoriasis, etc.

Increasing the skin's HA content may prove to be a good strategy for countering the effects, in particular the (purely) visible effects, of skin aging. There are several strategies that can complement each other to achieve this object.

For example, injecting implants under the skin (subcutaneous or intradermal injection) in the form of a single-phase hydrogel comprising a gel composed of crosslinked hyaluronic acid and a physiologically acceptable salt thereof, is one solution proposed (see, for example, WO 2008/068297 A1). However, this involves an impractical method of administration requiring skilled personnel (subcutaneous or intradermal injection).

Thus, to overcome this difficulty of administration, numerous topical compositions for increasing HA content in the skin are proposed.

For example, EP2868313 discloses a composition for topical cosmetic and/or dermatological application comprising a combination of low molecular weight hyaluronic acid and ascorbic acid, and optionally an oligosaccharide.

US-2011/0003020 describes a composition for topical application allowing the treatment of skin aging. The composition comprises a pharmaceutically effective amount of estradiol (i.e. oestradiol), estriol (i.e. oestriol), hyaluronic acid and green tea extract, and optionally an effective amount of ascorbic acid.

FR3084592 describes a composition comprising HA for combating all the signs of aging (wrinkles, loss of firmness and elasticity, loss of radiance, etc.) by stimulating cell regeneration.

However, when these solutions involve enriching in HA by direct supply thereof, they do not allow the natural synthesis of HA to be stimulated. This may thus result in increased dependence on these (topical) compositions to compensate for the lack of HA, which is synthesized less and less naturally.

Thus, the prior art lacks a variety of solutions for inhibiting HA degradation or stimulating its synthesis, or even for combining the two approaches to amplify the effect.

The object of the present invention is to propose a composition which can meet this need.

### SUMMARY OF THE INVENTION

The subject of the present invention therefore relates to a cosmetic use of baicalin or a derivative thereof, optionally in combination with chlorogenic acid or a derivative thereof, in the cellular synthesis of hyaluronic acid, in particular in the skin.

The subject of the present invention also relates to a composition comprising baicalin or a derivative thereof, optionally in combination with chlorogenic acid or a derivative thereof, for use in the cellular synthesis of hyaluronic acid and/or in an immunocutaneous response.

The subject of the present invention thus relates to a composition comprising baicalin or a derivative thereof in combination with chlorogenic acid or a derivative thereof.

The subject of the present invention also relates to the cosmetic use of a composition comprising baicalin or a derivative thereof in combination with chlorogenic acid or a derivative thereof, according to the invention, to improve the general appearance of the skin.

The subject of the present invention also relates to a cosmetic composition comprising baicalin or a derivative thereof in combination with chlorogenic acid or a derivative thereof.

The subject of the present invention also relates to a dietary supplement comprising baicalin or a derivative thereof, more particularly in combination with chlorogenic acid or a derivative thereof.

The subject of the present invention also relates to a composition comprising baicalin or a derivative thereof in combination with chlorogenic acid or a derivative thereof for use as a medicament.

The subject of the present invention thus also relates to a composition as described above for use in the cellular synthesis of beta-defensin.

The subject of the present invention moreover relates to a composition as described above for use in the treatment of a disorder involving cellular synthesis of beta-defensin. More specifically, the subject of the present invention relates to a composition for use in the treatment of a disorder involving cellular synthesis of beta-defensin, characterized in that the disorder is a skin disorder chosen from microbial infection, restrictive dermopathy, dermatitis, such as atopic dermatitis, psoriasis and acne.

### Definitions

Baicalin is a natural molecule found in certain plants such as Scutellaria baicalensis Georgi. Its structure is as follows (1):

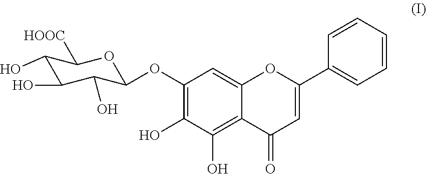 [SMILES: O=C(O)C1OC(Oc2cc3oc(-c4ccccc4)cc(=O)c3c(O)c2O)C(O)C(O)C1O]

Scutellaria baicalensis Georgi is a Chinese plant that has been traditionally used for centuries for treating inflammatory problems. Scutellaria baicalensis Georgi root extract is rich in flavonoid derivatives such as baicalin, baicalein and wogonin. Baicalin is already known for its cosmetic activity on the skin (FR3075050).

The term “baicalin derivatives” is understood in the context of the present invention to mean the same derivatives as those described in FR3075050 or WO 2005/044281A1, namely compounds covered by formula (IA) below:

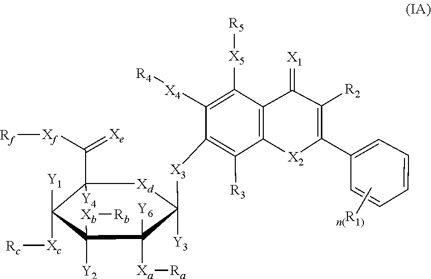

in which:
   each X₁, X₂, X₃, X₄, X₅, Xₐ, X_{b}, X_{c}, X_{d}, Xₑ and X_{f} independently denotes O or S;
   each Y₁, Y₂, Y₃, Y₄, Y₅ and Y₆ independently denotes H or a (C₁-C₁₀)alkyl radical, in particular methyl;
   each R₄, R₅, Rₐ, R_{b} and R_{c} independently denotes H, a (C₁-C₁₀)alkyl radical optionally substituted with 1 to 5 groups R_{y}, or a (C₁-C₁₀)alkyl-O-(C₁-C₁₀)alkyl radical, each (C₁-C₁₀)alkyl radical possibly being substituted with 1 to 5 groups R_{y};
   each R_{y}, independently, denotes R_{q} or a radical -(C₂-C₁₀)alkenyl, -(C₂-C₁₀)alkynyl, -(C₃-C₁₀)cycloalkyl, -(C₈-C₁₄)bicycloalkyl, -(C₈-C₁₄)tricycloalkyl, -(C₅-C₁₀)cycloalkenyl, -(C₈-C₁₄)tricycloalkenyl, phenyl, naphthyl, -(C₁₄)aryl, each of which may be substituted with one or more radicals R_{z};
   each R₁, R₂, R₃ independently, denotes R_{q} or a radical -(C₂-C₁₀)alkenyl, -(C₂-C₁₀)alkynyl, -(C₃-C₁₀)cycloalkyl, -(C₈-C₁₄)bicycloalkyl, -(C₈-C₁₄)tricycloalkyl, -(C₅-C₁₀)cycloalkenyl, -(C₈-C₁₄)tricycloalkenyl, phenyl, naphthyl, -(C₁₄)aryl, each of which may be substituted with one or more radicals R_{z};
   R_{f} is H, (C₁-C₁₂)alkyl optionally substituted with 1 to 5 radicals R_{y}, (C₁-C₁₂)alkyl-O—(C₁-C₁₂)alkyl, each (C₁-C₁₂)alkyl radical possibly being substituted with 1 to 5 groups R_{y};
   each R_{q}, independently, is CN, OH, halogen, N₃, NO₂, N(R_{z})₂, ═NR_{z}, CH═NR_{z}, NR_{z}OH, OR_{z}, COR_{z}, C(O)R_{z}, O(CO)OR_{z}, SR_{z}, S(O)R_{z} or S(O)₂R_{z};
   each R_{z}, independently, is -(C₁-C₆)alkyl, -(C₂-C₆)alkenyl, -(C₃-C₈)cycloalkyl, -(C₃-C₈)cycloalkenyl, phenyl, a heterocycle bearing 3 to 5 branches, CH(halo)₂ or C(halo)₃; and
   n is 0, 1, 2, 3, 4 or 5;
and also the salts thereof, the optical isomers thereof and/or the diastereoisomers thereof.

Some compounds of formula (IA) may contain asymmetric centers and exist in different enantiomeric and diastereoisomeric forms. A compound of formula (IA) may be in the form of an optical isomer or a diastereoisomer. According to the invention, the compounds of formula (IA) also comprise their optical isomeric or diastereoisomeric forms and mixtures thereof, including racemic mixtures.

The term “-(C₁-C₁₀)alkyl” means a saturated, linear or branched, non-cyclic hydrocarbon-based chain containing from 1 to 10 carbon atoms. Examples of saturated linear -(C₁-C₁₀)alkyl radicals include methyl, ethyl, n-propyl, n-butyl, n-pentyl, n-hexyl, n-heptyl, n-octyl, n-nonyl and n-decyl. As examples of branched saturated -(C₁-C₁₀)alkyl radicals, mention may be made of isopropyl, sec-butyl, isobutyl, tert-butyl, isopentyl, 2-methylbutyl, 3-methylbutyl, 2,2-dimethylbutyl, 2,3-dimethylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl and 3,3-diethylhexyl.

The term “-(C₂-C₁₀)alkenyl” means an unsaturated, linear or branched non-cyclic hydrocarbon-based chain containing from 2 to 10 carbon atoms and comprising at least one carbon-carbon double bond. As examples of (C₂-C₁₀)alkenyl radicals, mention may be made of: 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl.

The term “-(C₂-C₁₀)alkynyl” means an unsaturated, linear or branched non-cyclic hydrocarbon-based chain containing from 2 to 10 carbon atoms and comprising at least one carbon-carbon triple bond. As examples of (C₁-C₁₀)alkynyl radicals, mention may be made of: acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl.

The term “-(C₃-C₁₀)cycloalkyl” means a saturated hydrocarbon-based ring containing from 3 to 10 carbon atoms. As examples of -(C₃-C₁₀)cycloalkyl radicals, mention may be made of: cyclopropyl, cyclobutyl, cyclopentyl, cyclohexyl, cycloheptyl, cyclooctyl, cyclononyl and cyclodecyl.

The term “-(C₈-C₁₄)bicycloalkyl” means a hydrocarbon-based bicycle containing from 8 to 14 carbon atoms and at least one saturated cycloalkyl ring. As examples of -(C₈-C₁₄)bicycloalkyl radicals, mention may be made of: indanyl, 1,2,3,4-tetrahydronaphthyl, 5,6,7,8-tetrahydronaphthyl and perhydronaphthyl.

The term “-(C₈-C₁₄)tricycloalkyl” means a hydrocarbon-based tricycle containing from 8 to 14 carbon atoms and at least one saturated cycloalkyl ring. As examples of -(C₈-C₁₄)tricycloalkyl radicals, mention may be made of: pyrenyl, 1,2,3,4-tetrahydroanthracenyl, perhydroanthracenyl, aceanthrenyl, 1,2,3,4-tetrahydropenanthrenyl, 5,6,7,8-tetrahydrophenanthrenyl, perhydrophenanthrenyl.

The term “-(C₅-C₁₀)cycloalkenyl” means a non-aromatic hydrocarbon-based cyclic radical bearing at least one carbon-carbon double bond in the ring system and from 5 to 10 carbon atoms. As examples of -(C₅-C₁₀)cycloalkenyl radicals, mention may be made of: cyclopentadienyl, cyclohexenyl, cyclohexadienyl, cycloheptenyl, cycloheptadienyl, cycloheptatrienyl, cyclooctenyl, cyclooctadienyl, cyclooctatrienyl, cyclooctatetraenyl, cyclononenyl, cyclononadienyl, cyclodecenyl, cyclodecadienyl.

The term “-(C₈-C₁₄)bicycloalkenyl” means a hydrocarbon-based bicycle bearing at least one carbon-carbon double bond in each ring and from 8 to 14 carbon atoms. As examples of -(C₈-C₁₄)bicycloalkenyl radicals, mention may be made of: indenyl, pentalenyl, naphthalenyl, azulenyl, heptalenyl, 1,2,7,8-tetrahydronaphthalenyl.

The term “-(C₈-C₁₄)tricycloalkenyl” means a hydrocarbon-based tricycle bearing at least one carbon-carbon double bond in each ring and from 8 to 14 carbon atoms. As examples of -(C₈-C₁₄)tricycloalkenyl radicals, mention may be made of: anthracenyl, phenalenyl, acenaphthalenyl, as-indacenyl, s-indacenyl.

The term “-(C₁₄)aryl” means an aromatic carbocycle having 14 branches, such as anthryl and phenanthryl.

The term “heterocycle bearing 3 to 5 branches” means a saturated, unsaturated, aromatic or non-aromatic heteromonocycle bearing 3 to 5 branches containing carbon atoms and heteroatoms. A heterocycle bearing 3 or 4 branches may include up to 3 heteroatoms and a heterocycle bearing 5 branches may include up to 4 heteroatoms. Each heteroatom is independently chosen from a nitrogen, which may be quaternized, oxygen, and sulfur, including sulfoxide and sulfone.

The heterocycle may be attached via any heteroatom or carbon atom. As examples of heterocycles bearing 3-5 branches, mention may be made of: furyl, thiophenyl, pyrrolyl, oxazolyl, imidazolyl, thiazolyl, isoxazolyl, pyrazolyl, isothiazolyl, triazinyl, pyrrolidinonyl, pyrrolidinyl, hydantoinyl, oxiranyl, oxetanyl, tetrahydrofuryl and tetrahydrothiophenyl.

The term “halo” means a halogen atom such as F (fluorine), Cl (chlorine), Br (bromine) and I (iodine).

The term “—CH(halo)₂” means a methyl group in which two of the hydrogens are replaced with a halogen atom. Examples that may be mentioned include: —CHF₂, —CHCI₂, —CHBr₂, —CHBrCl, —CHClI and —CHI₂.

The term “—CH(halo)₃” means a methyl group in which three of the hydrogens are replaced with a halogen atom. Examples that may be mentioned include: —CF₃, —CF₂C, —CC₃, —CBr₃, —CFBr₂ and —C₃.

The term “salts of the compounds of formula (I)” means a salt formed with an inorganic or organic acid or else an inorganic or organic base. As examples of acid salts, mention may be made of the sulfate, citrate, acetate, oxalate, chloride, bromide, iodide, nitrate, bisulfate, phosphate, isonicotinate, lactate, salicylate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e. 1,1′-methylenebis(2-hydroxy-3-naphthoate)) salts.

As examples of base salts, mention may be made of alkali metal hydroxides such as sodium, potassium and lithium hydroxides; alkaline-earth metal hydroxides such as calcium and magnesium hydroxides; hydroxides of other metals, such as aluminum and zinc hydroxides; ammonia and organic amines such as unsubstituted or hydroxy-substituted mono-, di- or tri-alkylamines; dicyclohexylamines; tributylamines; pyridine; N-methyl-N-ethylamine; diethylamine; triethylamine; mono-, bis- or tris(2-hydroxyalkylamines) such as mono-, bis- or tris-(2-hydroxyethyl)amine, 2-hydroxy-tert-butylamine, or tris(hydroxymethyl)methylamine, N,N-di-alkyl-N-(hydroxyalkyl)amines, such as N,N-dimethyl-N-(2-hydroxyethyl)amine or tris(2-hydroxyethyl)amine; N-methyl-D-glucamine; and amino acids such as arginine and lysine.

According to a preferred form of the invention, at least one of the radicals X₁, X₂, X₃, X₄, X₅, Xₐ, X_{b}, X_{c}, X_{d}, Xₑ and X_{f} is O.

According to a preferred form of the invention, at least one of the radicals Y₁, Y₂, Y₃, Y₄, Y₅, Y₆ independently denotes H.

According to a preferred form of the invention, at least one of the radicals Y₁, Y₂, Y₃, Y₄, Y₆ independently denotes CH₃.

According to a preferred form of the invention, R₁ denotes H or CH₃.

According to a preferred form of the invention, n is equal to 5.

According to a particularly preferred form, the composition of the invention comprises baicalin corresponding to the general formula (I) above or a plant extract containing same.

This compound is notably described in patent application WO 2008/140440, notably in the form of a solution. Baicalin may be used in the form of a solution comprising an alkyl glycol containing from 2 to 7 carbon atoms, a polyol ether, and at least one antioxidant. Such an organic compound may be obtained as described in EP 1 400 579 (US 2004/0067894) relating to the synthesis of tetrahydroxyflavones, the general formula of which comprises baicalin.

The baicalin may be used in the form of an extract of plant origin. Baicalin is a polyphenol (flavone) extracted notably from scullcap root, in particular of Scutellaria baicalensis, having the INCI name: Scutellaria baicalensis root extract. It originates from traditional Chinese medicine. The various methods for preparing the extracts are described in patent application WO 2005/044281.

The term “cosmetic use” is understood in the context of the present invention to mean use involving purely visual effects, i.e. not involving a therapeutic effect and thus having an effect on skin radiance, a visual antiaging effect, an improvement in skin sensitivity, a visual treatment of acne, etc. Specifically, depending on the degree of hyaluronic acid deficiency, the skin may present benign, non-pathological symptoms, i.e. purely visual. In this context, the use is restricted to cosmetic improvement.

The term “skin sensitivity improvement” is understood in the context of the present invention to mean an improvement in the physiological characteristics of the skin, making it less sensitive to external attacking factors (light or other electromagnetic waves, pollution, cold, dryness, sources of free radicals, etc.) involving premature aging of the skin, favoring the appearance of wrinkles, fine lines or even benign cutaneous dryness. It should be noted that, depending on the amount of product according to the invention administered and the disorder treated, a therapeutic effect may also be achieved. The amounts to be administered depend on the nature of the disorder and the patient's characteristics (age, past or present pathologies, etc.), which the practitioner will be capable of determining.

Cosmetic use may simply be achieved by limiting the amounts administered and/or the modes of administration of the compositions according to the present invention. For example, daily topical administration (i.e. once a day) of a small amount (for example less than or equal to 0.01 g/dm³, or even less than or equal to 0.001 g/dm³, of composition applied in a layer of not more than 1 mm, such as 0.1 mm, on the skin).

The term “cosmetic and/or dermatological product” thus means any product which allows a composition to be constituted which may be applied cosmetically and/or dermatologically, i.e. a composition which may, for example, be applied to the skin or which may be used for cosmetically and/or dermatologically treating the skin.

Chlorogenic acid, an ester formed between caffeic acid and L-quinic acid, is a widespread phenolic compound found in foods such as coffee, apples, pears, etc. There are several isomers and salts of chlorogenic acid (e.g., the “trans” or “cis” forms, basic forms or salts of chlorogenic acid).

Chlorogenic acid (CA) may thus be represented in a trans form according to formula (II):

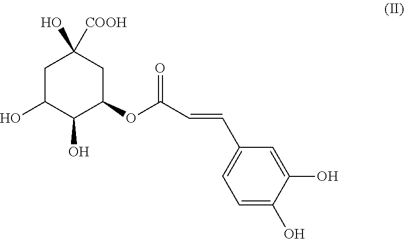 [SMILES: O=C(/C=C/c1ccc(O)c(O)c1)O[C@@H]1C[C@@](O)(C(=O)O)CC(O)[C@@H]1O]

The term “chlorogenic acid derivatives” is understood in the context of the present invention to mean the same derivatives as those described in US 2004/097584 A1, namely compounds covered by formula (IIA) below:

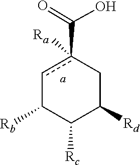

in which:
   “a” represents either a single bond or a double bond and Rₐ is present only in the case where “a” represents a single bond, and in which at least one of the functional groups Rₐ, R_{b}, R_{c} and R_{d}, preferably at least R_{d}, represents a functional group represented by formula IIB below (also referred to hereinbelow as subst.IIB):

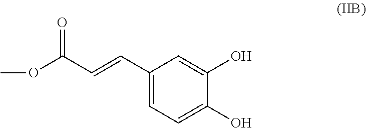

and the remainder of said functional groups Rₐ, R_{b}, R_{c} and R_{d} are independently chosen from the group formed by —OH, and other substituents analogous to caffeic acid fragments—which are abundant in plant material and may be separated by intestinal flora. It should be noted that the compound of formula IIA has the 1α, 3α, 4α, 5β-hydroxy configuration.

Preferred functional analogs of chlorogenic acid in a preparation according to the invention are 4-caffeoylquinic acid, neochlorogenic acid (3-caffeoylquinic acid), dicaffeoylquinic acids in which positions 1 and 5 are substituted (1,5-dicaffeoylquinic acid), isochlorogenic acids (i.e. dicaffeoylquinic acids substituted in positions 3 and 4, positions 3 and 5 or positions 3, 4 and 5 (3,4,5-ticaffeoylquinic acid), kryptochlorogenic acid (4-O-caffeoylquinic acid, cynarine (1,3-O-caffeoylquinic acid), 1,3,4,5-O-tetracaffeoylquinic acid and caffeoylshikimic acids, in particular 5-O-caffeoylshikimic acid.

With reference to formula (IIA), these analogs may have the following chemical structures (Table 1):

| Compounds | Rₐ | R_{b} | R_{c} | R_{d} |
|---|---|---|---|---|
| (−)-quinic acid | OH | OH | OH | OH |
| 1-O-caffeoylquinic acid | subst.IIB | OH | OH | OH |
| chlorogenic acid (5-O- | OH | OH | OH | subst.IIB |
| caffeoylquinic acid) | | | | |
| kryptochlorogenic acid | OH | OH | subst.IIB | OH |
| neochlorogenic acid | OH | subst.IIB | OH | OH |
| cynarine (1,3-O-dicaffeoylquinic | subst.IIB | subst.IIB | OH | OH |
| acid) | | | | |
| 1,5-O-dicaffeoylquinic acid | subst.IIB | OH | OH | subst.IIB |
| 3,4-O-dicaffeoylquinic acid | OH | subst.IIB | OH | OH |
| 3,5-O-dicaffeoylquinic acid | OH | subst.IIB | OH | subst.IIB |
| 4,5-O-dicaffeoylquinic acid | OH | OH | subst.IIB | subst.IIB |
| 1,2,3,4-O-tetracaffeoylquinic acid | subst.IIB | subst.IIB | subst.IIB | subst.IIB |

Chlorogenic acid an derivatives thereof may be synthesized by applying one of the methods known in the art, such as the method described by Panizzi et al. (Gazz. Chim. Ital, 86, 913, (1956)). Alternative sources for chlorogenic acid and derivatives thereof include, inter alia, Vaccinium myrtillus extracts, potato extracts (pressed liquor) and sunflower extracts.

The term “synthesis, preferentially cutaneous, of hyaluronic acid” is understood in the context of the present invention to mean synthesis of hyaluronic acid by natural pathways, in particular those found in the skin.

The term “immunocutaneous response” is understood in the context of the present invention to mean the implementation of various immune defenses in the skin, for example Langerhans cells and epidermal T cells (cf. Ref. 19).

### DETAILED DESCRIPTION

### Baicalin

The subject of the present invention thus relates to a composition comprising baicalin or a derivative thereof.

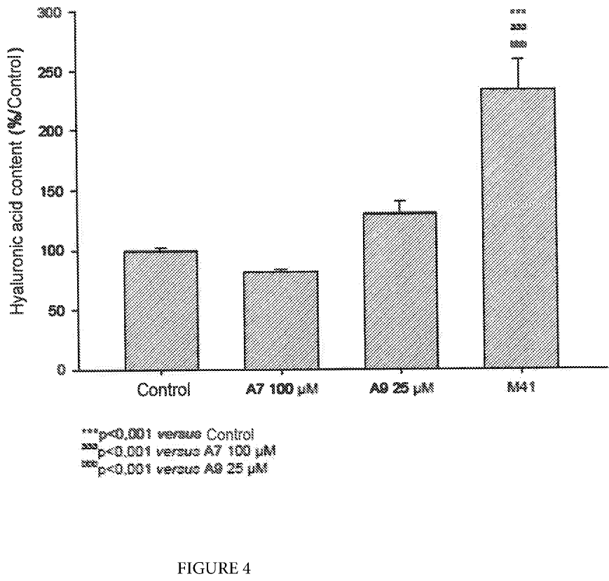

Specifically, the literature does not appear to describe any effect of baicalin on HA stimulation. However, it has been possible to finbd such activity in the context of the present invention (cf. FIG. 4, “product 9”).

Admittedly, it is known that baicalin has an anti-inflammatory effect based on inhibition of lipoxygenase and arachidonate metabolism (refs. 2, 3, 8 and 9). This anti-inflammatory effect is reinforced by its antioxidant activity, more specifically the trapping of hydroxyl, superoxide and DPPH free radicals, which is greater than that of alpha-tocopherol (refs. 4, 5, 6 and 7), and also by the inhibition of nitric oxide producrieftion.

Baicalin is also known to have anti-allergic properties by virtue of its ability to bind chemokines and limit their biological functions (refs. 1 and 11). By binding to antibodies, it inhibits the IGE antibody-mediated reaction and histamine release (refs. 8, 9 and 10). The effects of baicalin against UVA-induced photo-aging have been demonstrated. Treatment with baicalin effectively protects human fibroblasts against these UVA-induced aging reactions, suggesting that the underlying mechanism involves inhibition of oxidative damage and regulation of senescence-related gene expression, including those encoding p53, p66 (Shc) and p16 (Ref. 17).

Patents US 2018/0369111, US 2020/0297612, U.S. Pat. No. 9,072,919 B2, FR 3 075 050 B1 and U.S. Pat. No. 9,018,177 B2 also describe the use of baicalin in cosmetics.

However, none of these documents directly show any effect of baicalin on increasing HA content in skin cells such as fibroblasts. However, when used at a safe level (e.g. 25 μM), baicalin appears to improve HA content relative to a control test (cf. FIG. 4, product 9).

The subject of the present invention thus relates to baicalin or a derivative thereof, for use in the cellular synthesis of hyaluronic acid, in particular in the skin.

The subject of the present invention thus also relates to (depending on the amount administered) baicalin or a derivative thereof, for use in an immunocutaneous response. This activity in itself is relatively surprising given that baicalin was known for its anti-inflammatory and anti-allergic properties, effects which are often deleterious with the stimulation of an immunological response.

However, the effect of baicalin on cellular synthesis of hyaluronic acid appears to indicate that baicalin or a derivative thereof may also be used for improving an immunocutaneous response, when administered in sufficient amount.

### Chlorogenic Acid (CA)

The polyphenol chlorogenic acid has powerful antioxidant properties (Ref. 12, Ref. 13) and is recognized for its protein-protective action. Eucommia ulmoides leaves afford an extract with a high chlorogenic acid content. This plant is used in traditional Chinese medicine for treating the liver and kidneys.

In addition to its antioxidant properties, chlorogenic acid has been shown to have an anti-glycation effect (Ref. 14). This molecule thus allows the skin's elastic network to be protected from degradation, and maintains the expression of fibrillin-1, a glycoprotein associated with oxytalan fibers and involved in the formation of elastic fibers.

Moreover, one study has shown that chlorogenic acid is involved in melanogenesis and affects tyrosinase activity. (Ref. 15). This bioactive compound could be used in cosmetic applications relating to skin lightening.

Another study has highlighted the healing properties of chlorogenic acid by virtue of its ability to boost collagen synthesis (Ref. 16).

Finally, a recent study (Ref. 18) demonstrated the anti-aging properties of CA by improving the hyaluronic acid (HA) content of skin cells. However, these results could not be reproduced at a tested level that might be safe for health (for example 100 μM; cf. FIG. 4, product 7).

### Combination of Baicalin and Chlorogenic Acid (CA)

Surprisingly, the combination of baicalin and chlorogenic acid allowed hyaluronic acid to be synthesized at a much higher content than expected, demonstrating the synergistic effect of these two compounds (cf. FIG. 4, product “M41”).

The subject of the present invention thus relates to a combination of baicalin or a derivative thereof with chlorogenic acid or a derivative thereof, for use in the cellular synthesis of hyaluronic acid, in particular in the skin.

The subject of the present invention thus also relates to (depending on the amount administered) a combination of baicalin or a derivative thereof with chlorogenic acid or a derivative thereof, for use in an immunocutaneous response.

### Compositions According to the Present Invention

In general, a “composition according to the present invention” or equivalent expression (such as “formulation of the invention”) in the context of the present invention thus generally corresponds to a composition:
of baicalin or a derivative thereof for use in the cellular synthesis of hyaluronic acid, in particular in the skin;
of baicalin or a derivative thereof for use in an immunocutaneous response;
of baicalin or a derivative thereof in combination with chlorogenic acid or a derivative thereof, in a general manner;
of baicalin or a derivative thereof in combination with chlorogenic acid or a derivative thereof, more particularly for use in the cellular synthesis of hyaluronic acid, in particular in the skin; and/or
of baicalin or a derivative thereof in combination with chlorogenic acid or a derivative thereof, more particularly for use in an immunocutaneous response.

The composition according to the present invention may thus serve for esthetic and/or reparative purposes.

In one particular embodiment, the subject of the present invention relates to a composition comprising baicalin or a derivative thereof, for use in the cellular synthesis of hyaluronic acid (HA), in particular in the skin. Specifically, the inventors have found that when used at safe levels (for example 25 μM), baicalin appears to improve HA content relative to a control test (cf. FIG. 4, product 9).

In one particular embodiment, the subject of the present invention relates to a composition comprising baicalin or a derivative thereof in combination with chlorogenic acid or a derivative thereof, in the cellular synthesis of hyaluronic acid, in particular in the skin. Specifically, the inventors have found that when used at safe levels (for example 25 μM for baicalin and 100 μM for chlorogenic acid), the combination of baicalin with chlorogenic acid significantly improves HA content compared to a test comprising baicalin alone, thus revealing a surprising effect. Specifically, synergism was found in the production of HA in the skin when a composition comprising baicalin or a derivative thereof and chlorogenic acid or a derivative thereof were applied. Such synergism may indeed be found in the context of the present invention (cf. FIG. 4, product M41).

The subject of the present invention thus relates to a composition according to the present invention, characterized in that the ratio between the dry mass amount of baicalin or derivative thereof and the dry mass amount of chlorogenic acid or derivative thereof, where appropriate, is between 1:1 and 1:10 respectively, between 1:1.5 and 1:8 respectively, between 1:2 and 1:7 respectively, between 1:2.5 and 1:6 respectively, preferentially between 1:3 and 1:5 respectively, more preferentially between 1:3.5 and 1:4.5 respectively, such as 1:4, respectively.

The subject of the present invention thus relates to a formulation according to the invention, characterized in that the concentration of baicalin or derivative thereof in the composition is advantageously less than or equal to 50 micromol, preferably less than or equal to 35 micromol, less than or equal to 25 micromol, less than or equal to 20 micromol, less than or equal to 15 micromol, less than or equal to 10 micromol, or even less than or equal to 5 micromol of baicalin or derivative thereof per dm³ of composition.

The subject of the present invention thus concerns a formulation according to the invention, characterized in that the concentration of chlorogenic acid or derivative thereof, where appropriate, in the composition is advantageously less than 200, preferentially less than or equal to 150 micromol, less than or equal to 100 micromol, less than or equal to 90 micromol, less than or equal to 80 micromol, less than or equal to 75 micromol, less than or equal to 70 micromol, less than or equal to 65 micromol, less than or equal to 60 micromol, less than or equal to 55 micromol, less than or equal to 50 micromol, less than or equal to 45 micromol, less than or equal to 40 micromol, less than or equal to 35 micromol, less than or equal to 30 micromol, less than or equal to 25 micromol, less than or equal to 20 micromol, less than or equal to 15 micromol, less than or equal to 10 micromol, or even less than or equal to 5 micromol of chlorogenic acid or a derivative thereof per dm³ of composition.

The subject of the present invention thus relates to a composition according to the present invention, characterized in that the concentration of baicalin or derivative thereof in the composition is advantageously less than 50 micromol, preferentially less than or equal to 25 micromol, of baicalin or derivative thereof per dm³ of composition and, where appropriate, in that the concentration of chlorogenic acid or derivative thereof in the composition is advantageously less than 200 micromol, preferentially less than or equal to 100 micromol, of chlorogenic acid or derivative thereof per dm³ of composition.

The compositions according to the invention may also contain formulation aids known to those skilled in the art, for instance stabilizers, preserving agents or pH adjusters.

The subject of the present invention thus relates to a composition according to the present invention, characterized in that said composition may comprise at least one solvent and a pH stabilizer.

The subject of the present invention thus relates to a composition according to the present invention, characterized in that said composition is suitable for the physiological medium. The physiologically acceptable medium may thus consist essentially of water, or of an isotonic aqueous solution. It may also contain buffering agents, viscosity agents and/or antioxidants.

The compositions according to the invention may notably take the form of aqueous, aqueous-alcoholic or oily solutions, lotion- or serum-type dispersions, anhydrous or oily gels, or milk-type emulsions of liquid or semi-liquid consistency, obtained by dispersing a fatty phase in an aqueous phase (0/W) or vice versa (W/G), suspensions or emulsions of soft, semisolid or solid consistency such as creams, gels, microemulsions, or even microcapsules, microparticles, or ionic and/or nonionic vesicular dispersions. These compositions are prepared according to the usual methods.

The subject of the present invention thus relates to a composition according to the present invention, characterized in that said composition may comprise at least one of the compounds chosen from the following list: sodium hyaluronate, hydrolyzed sodium hyaluronate, sodium carboxymethyl beta-glucan, inulin, an alpha-glucan oligosaccharide, arginine and pentylene glycol.

The subject of the present invention thus relates to a composition comprising baicalin or a derivative thereof, in combination with chlorogenic acid or a derivative thereof, characterized in that said composition comprises at least one, at least two, at least three, at least four, at least five, at least six, or all seven, of the compounds chosen from the following list: sodium hyaluronate, hydrolyzed sodium hyaluronate, sodium carboxymethyl beta-glucan, inulin, an alpha-glucan oligosaccharide, arginine and pentylene glycol.

The subject of the present invention also relates to a composition, for example a concentrated composition as a “premix”, comprising baicalin or a derivative thereof, more particularly in combination with chlorogenic acid or a derivative thereof, more particularly characterized in that said composition comprises between 14% and 70% by mass of water, preferentially between 40% and 60% by mass of water, such as 50% plus or minus 2% by mass of water.

The subject of the present invention thus relates to a composition, for example a concentrated composition as a “premix”, comprising baicalin or a derivative thereof, more particularly in combination with chlorogenic acid or a derivative thereof, more particularly characterized in that said composition comprises between 0.0001% and 6% by mass, preferentially 2% by mass, of baicalin or a derivative thereof.

The subject of the present invention thus relates to a composition, for example a concentrated composition as a “premix”, comprising baicalin or a derivative thereof, more particularly in combination with chlorogenic acid or a derivative thereof, more particularly characterized in that said composition comprises, where appropriate, between 0.0004% and 20% by mass, preferentially 8% by mass, of chlorogenic acid or a derivative thereof.

The subject of the present invention also relates to a composition, for example a concentrated composition as a “premix”, comprising baicalin or a derivative thereof, more particularly in combination with chlorogenic acid or a derivative thereof, more particularly characterized in that said composition comprises, where appropriate, between 0.0004% and 2% by mass, preferentially 0.8% by mass, of sodium carboxymethyl beta-glucan.

The subject of the present invention also relates to a composition, for example a concentrated composition as a “premix”, comprising baicalin or a derivative thereof, more particularly in combination with chlorogenic acid or a derivative thereof, more particularly characterized in that said composition comprises, where appropriate, between 0.0008% and 30% by mass, preferentially 16% by mass, of inulin.

The subject of the present invention also relates to a composition, for example a concentrated composition as a “premix”, comprising baicalin or a derivative thereof, more particularly in combination with chlorogenic acid or a derivative thereof, more particularly characterized in that said composition comprises, where appropriate, between 0.00002% and 10% by mass, preferentially 4% by mass, of alpha-glucan oligosaccharide.

The subject of the present invention also relates to a composition, for example a concentrated composition as a “premix”, comprising baicalin or a derivative thereof, more particularly in combination with chlorogenic acid or a derivative thereof, more particularly characterized in that said composition comprises, where appropriate, between 0.001% and 30% by mass, preferentially 15% by mass, of pentylene glycol.

The subject of the present invention also relates to a composition, for example a concentrated composition as a “premix”, comprising baicalin or a derivative thereof, more particularly in combination with chlorogenic acid or a derivative thereof, more particularly characterized in that said composition comprises, where appropriate, between 0.001% and 10% by mass, preferentially 4.3% by mass, of arginine.

The subject of the present invention also relates to a composition, for example a concentrated composition as a “premix”, comprising baicalin or a derivative thereof, more particularly in combination with chlorogenic acid or a derivative thereof, more particularly characterized in that said composition comprises:
between 0.0001% and 6% by mass, preferentially 2% by mass, of baicalin or derivative thereof;
where appropriate, between 0.0004% and 20% by mass, preferentially 8% by mass, of chlorogenic acid or derivative thereof;
where appropriate, between 0.0004% and 2% by mass, preferentially 0.8% by mass, of sodium carboxymethyl beta-glucan;
where appropriate, between 0.0008% and 30% by mass, preferentially 16% by mass, of inulin; and
where appropriate, between 0.00002% and 10% by mass, preferentially 4% by mass, of alpha-glucan oligosaccharide.

The subject of the present invention also relates to a composition, for example a concentrated composition as a “premix”, comprising baicalin or a derivative thereof in combination with chlorogenic acid or a derivative thereof, characterized in that said composition comprises:
2% by mass of baicalin or derivative thereof;
8% by mass of chlorogenic acid or derivative thereof;
0.8% by mass of sodium carboxymethyl beta-glucan;
16% by mass of inulin;
4% by mass of alpha-glucan oligosaccharide;
optionally 15% by mass of pentylene glycol; and
optionally 4.3% by mass of arginine.

The subject of the present invention also relates to a composition, for example a concentrated composition as a “premix”, comprising baicalin or a derivative thereof, more particularly in combination with chlorogenic acid or a derivative thereof, characterized in that said composition comprises:
between 0.0001% and 1% by mass of baicalin or derivative thereof;
between 0.0004% and 4% by mass of chlorogenic acid or derivative thereof;
between 0.0004% and 0.8% by mass of sodium carboxymethyl beta-glucan;
between 0.0008% and 8% by mass of inulin; and/or
between 0.00002% and 2% by mass of alpha-glucan oligosaccharide.

The subject of the present invention also relates to a composition, for example a concentrated composition as a “premix”, comprising baicalin or a derivative thereof, more particularly in combination with chlorogenic acid or a derivative thereof, more particularly characterized in that said composition comprises:
between 0.001% and 0.8% by mass of baicalin or derivative thereof;
where appropriate, between 0.01% and 3% by mass of chlorogenic acid or derivative thereof;
where appropriate, between 0.001% and 0.6% by mass of sodium carboxymethyl beta-glucan;
where appropriate, between 0.0008% and 6% by mass of inulin; and
where appropriate, between 0.0002% and 1.5% by mass of alpha-glucan oligosaccharide.

The subject of the present invention also relates to a composition, for example a concentrated composition as a “premix”, comprising baicalin or a derivative thereof, more particularly in combination with chlorogenic acid or a derivative thereof, more particularly characterized in that said composition comprises:
between 0.01% and 0.5% by mass of baicalin or derivative thereof;
where appropriate, between 0.1% and 2.5% by mass of chlorogenic acid or derivative thereof;
where appropriate, between 0.01% and 0.5% by mass of sodium carboxymethyl beta-glucan;
where appropriate, between 0.008% and 5% by mass of inulin; and
where appropriate, between 0.002% and 1% by mass of alpha-glucan oligosaccharide.

The subject of the present invention also relates to a composition, for example a concentrated composition as a “premix”, comprising baicalin or a derivative thereof, more particularly in combination with chlorogenic acid or a derivative thereof, more particularly characterized in that said composition comprises:
between 0.1% and 0.4% by mass, preferentially 0.25% by mass, of baicalin or derivative thereof;
where appropriate, between 0.1% and 2% by mass, preferentially 1% by mass, of chlorogenic acid or derivative thereof;
where appropriate, between 0.01% and 0.4% by mass, preferentially 0.25% by mass, of sodium carboxymethyl beta-glucan;
where appropriate, between 1% and 3% by mass, preferentially 2.5% by mass, of a mixture of inulin and alpha-glucan oligosaccharide with a mass ratio of inulin:alpha-glucan oligosaccharide of between 7:1 and 9:3, such as 8:2.

The premixes according to the present invention may be diluted in a solvent such as water to contents of between 0.1% and 90% by mass, preferentially between 0.5% and 80%, between 1% and 70%, between 2% and 50%, between 5% and 30% or even between 10% and 15%, such as 12.5% by mass.

Thus, a 10% by mass dilution corresponds to 90 g of solvent, such as water, per 10 g of premix.

Preferably, sodium hyaluronate and hydrolyzed sodium hyaluronate may also be added to the solution obtained by diluting the premix, for example to a content of 0.2% by mass and 0.3% by mass, respectively.

The subject of the present invention thus concerns a composition comprising baicalin or a derivative thereof, more particularly in combination with chlorogenic acid or a derivative thereof, more particularly characterized in that said composition comprises, where appropriate, between 0.0001% and 1% by mass, preferentially 0.2% by mass, of a hyaluronate salt, such as sodium hyaluronate.

The subject of the present invention also relates to a composition comprising baicalin or a derivative thereof, more particularly in combination with chlorogenic acid or a derivative thereof, more particularly characterized in that said composition comprises, where appropriate, between 0.0001% and 1% by mass, preferentially 0.3% by mass, of a hydrolyzed hyaluronate salt, such as hydrolyzed sodium hyaluronate.

The subject of the present invention thus relates to a composition comprising baicalin or a derivative thereof, more particularly in combination with chlorogenic acid or a derivative thereof, more particularly characterized in that the composition is a cosmetic composition or a dietary supplement.

The subject of the present invention thus relates to a composition according to the present invention, characterized in that it may be a preferentially aqueous liquid composition, or a solid such as a powder.

The liquid composition according to the invention may be aqueous, oily and/or in emulsion form.

For example, the liquid composition according to the invention may be in the form of an oil-in-water or water-in-oil emulsion.

The composition according to the invention may be used in a cosmetic or pharmaceutical formulation, notably a dermatological formulation.

A liquid composition according to the invention may be used in a topical formulation, notably an external topical formulation.

The composition according to the invention may be used in a form that is suitable for administration by injection.

A liquid composition according to the invention may be used in an oral formulation, such as a dietary supplement.

The solid composition according to the invention may be used in an oral formulation, such as a dietary supplement.

The invention therefore also relates to a cosmetic treatment or prevention process for reducing or preventing the signs of aging of the skin or mucous membranes, and/or for restoring the volume of the body or face, characterized in that a composition according to the present invention is administered (topically, by injection or even systemically) to at least one cutaneous or subcutaneous tissue.

The invention also relates to a cosmetic treatment or prevention process for reducing signs of aging of the skin and/or mucous membranes, in particular for reducing wrinkles and fine lines and/or restoring volume to the face or body, or for increasing lip volume, in which a composition according to the present invention as defined previously is applied. The cosmetic treatment process according to the invention notably allows the prevention, erasure or attenuation of unsightly scar marks, for example resulting from acne; it also allows the restoration of body contours, for example in the context of breast implants, or facial contours, modified in the course of aging. In particular, the process according to the invention comprises the injection of said composition into at least one cutaneous or subcutaneous tissue.

The cosmetic and/or dermatological composition may be in any form suitable for its use.

### Topical Administration

The compound(s) used to constitute the cosmetic and/or dermatological product depend notably on the formulation chosen for the cosmetic and/or dermatological use of the composition of the present invention.

The topical formulations (cosmetic or medicinal) in accordance with the present invention may be in the forms conventionally used for topical application, i.e. in the form of gel, solution, suspension, lotion, cream, oil, emulsion (in particular in the form of cream or milk), microemulsion, paste, aerosol, ointment, foam, spot-on powder, serum (or essence), mask or ointment, containing usual compatible and cosmetically and/or pharmaceutically acceptable excipients and supports. They may also be in the form of controlled-release patches, tulles or dressings, or wipes soaked in a composition according to the invention.

These topical administration forms are prepared via techniques well known to those skilled in the art, and, for example, in the case of a cream, by dispersing a fatty phase in an aqueous phase to obtain an oil-in-water emulsion, or conversely to prepare a water-in-oil emulsion.

More specifically, the composition according to the invention may comprise one or more usual excipients, notably suitable for external topical administration, in particular dermatologically and cosmetologically acceptable excipients.

These excipients suitable for formulation are well known to those skilled in the art, and may be chosen, for example, from one or more of the compounds i to xv listed below:
i. antioxidant vitamins, such as vitamin E, for example tocopheryl acetate or tocotrienol; vitamin C or an analog thereof, natural polyphenols; the composition may thus optionally contain other anti-aging active agents, for example ascorbic acid or a derivative thereof;
ii. pH stabilizers;
iii. gelling and/or thickening agents, such as natural gums like xanthan gum; synthetic polymers, for example Carbopol-type polyacrylamides, crosslinked acrylates and cellulose derivatives;
iv. plant oils, for instance kukui oil, Calophyllum inophyllum oil, helichrysum oil, macadamia oil, Inca inchi oil, prickly pear oil, argan oil;
v. active principle carriers, such as cyclodextrins; liposomes, for example lecithin;
vi. emulsifiers and coemulsifiers, such as arachidyl glucoside; cetearyl glucoside; behenyl alcohol; cetearyl alcohol; cetostearyl alcohol; cetyl alcohol, steareth-2; steareth-21; isostearyl isostearate; isopropyl myristate, sorbitan esters and polysorbates;
vii. UV-screening agents, such as hydrophilic or lipophilic UV-A and UV-B sunscreens, chosen from benzophenone or a benzophenone derivative such as 2-hydroxy-4-methoxybenzophenone; a cinnamic acid ester, more particularly octyl methoxycinnamate and 2-ethylhexyl methoxycinnamate, or even a cyano-β,β-diphenylacrylate such as octocrylene, 4-methylbenzylidenecamphor, and dibenzoylmethane derivatives such as 4-isopropyldibenzoylmethane, t-butylmethoxydibenzoylmethane, and 4-methoxydibenzoylmethane; or such as UV-blocking pigments;
viii. preserving agents, such as phenoxyethanol, dehydroacetic acid; benzoic acid; potassium sorbate; sodium benzoate; methyl or ethyl para-hydroxybenzoates; the term “preserving agent” means any compound which enables the growth of microorganisms in a cosmetic and/or dermatological composition to be inhibited. These compounds are known to those skilled in the art. For example, they may be fungicidal and/or antibacterial agents; ix. humectants/moisturizing agents, such as glycerol; butylene glycol; propylene glycol;
x. exfoliating powders; fruit powders; or mattifying powders such as methyl methacrylate copolymer;
xi. emollients and surfactants, such as octyldodecanol; cetyl palmitate; cetearyl octanoate; isopropyl myristate; isononyl isononanoate; capric/caprylic triglycerides; hydrogenated polyisobutene; cetearyl isononanoate; polyglyceryl 3-diisostearate; cetyl phosphate; plant oils such as macadamia oil; xii. silicones such as dimethicone, cyclomethicone;
xiii. neutralizing agents;
xiv. solubilizers;
xv. dyes;
xvi. fragrances.

The cosmetic or dermatological composition according to the invention may notably comprise at least one cosmetically or dermatologically acceptable excipient. These excipients may be used alone or as mixtures to produce formulations for topical application to the skin. In accordance with the invention, the compositions may also comprise other active agents with a beneficial effect on the skin. By way of example, mention may be made of: a) hydrophilic compounds of plant origin such as extracts of argan, Ginkgo biloba, green tea, honey, oat germ and/or bran, wheat germ and/or bran, willow bark, witch hazel, Aloe vera, or mixtures thereof; b) silicon-based organic compounds; or c) soothing compounds such as allantoin, azulene, cornflower or sugars, notably sucrose.

In accordance with the invention, the compositions may thus comprise one or more chelating agents, one or more buffering agents, one or more binders, one or more thickeners, one or more UV-screening agents, one or more film-forming agents, one or more surfactants, one or more moisturizers, one or more stabilizers, one or more emulsion stabilizers, one or more absorbants, one or more humectants, one or more antioxidants, and/or one or more fragrances.

The term “chelating agent” means any compound which forms complexes with metal ions, modifying the stability and/or viscosity of a cosmetic and/or dermatological composition. These compounds are known to those skilled in the art. For example, the chelating agent may be chosen from the group comprising nitriloacetic acid, polyaminocarboxylic acid, ethylenediaminetetraacetic acid (EDTA) and salts thereof.

The term “buffering agent” means a compound which allows the pH of a cosmetic and/or dermatological composition to be stabilized. These compounds are known to those skilled in the art. For example, the buffering agent may be chosen from the group comprising aminomethylpropanol, citric acid, fumaric acid, orthophosphoric acid, sebacic acid (decanedioic acid), sodium citrate, sodium hydroxide, tartaric acid, tetrasodium pyrophosphate and triethylamine (TEA).

The term “binder” means a compound which allows the cohesion of a cosmetic composition to be increased. These compounds are known to those skilled in the art. For example, the binder may be chosen from the group comprising acrylate copolymers, Cyamopsis tetragonolubus (guar), hydroxyethylcellulose, isopropyl myristate, magnesium carbonate, mannitol, polyethylene glycol-90M, polydecene, PVP/eicosene copolymer, PVP/hexadecene copolymer, PVP/VA copolymer, sodium acrylate copolymer, sodium carboxymethyl beta-glucan copolymer, sodium carboxymethyl dextran, paraffin wax and xanthan gum.

The term “thickener” means any compound that is capable of increasing the viscosity of a cosmetic and/or dermatological composition. These compounds are known to those skilled in the art. For example, the thickener may be chosen from the group comprising natural polymers such as alginic acid and derivatives thereof, cellulose and derivatives thereof, scleroglucans, xanthan gum and gum arabic, synthetic carboxyvinyl polymers and copolymers which may also have an emulsifying function, for example acrylate, methacrylate, acrylamide and mixtures thereof, for example C10-30 alkyl acrylate/acrylate copolymer, or polymethyl methacrylate.

The term “UV-screening agent” means a compound which allows certain UV rays to be screened out. These compounds are known to those skilled in the art. For example, the UV-screening agent may be chosen from the group comprising benzophenone-3 (oxybenzone), benzophenone-4 (sulisobenzone), avobenzone, ethylhexyl methoxycinnamate, octyl methoxycinnamate and ethylhexyl salicylate.

The term “film-forming agent” means a compound which allows the formation of a continuous film on application of a cosmetic and/or dermatological composition. These compounds are known to those skilled in the art. For example, the film-forming agent may be chosen from the group comprising acrylamide/ammonium acrylate copolymer, acrylamide copolymer, acrylamidopyropyltrimonium chloride/acrylates copolymer, acrylates acrylates/C10-30 alkyl acrylate copolymer, white beeswax (E901), Cyamopsis tetragonolubus (guar), hydroxyethylcellulose, polyethylene terephthalate (PET), polyglyceryl methacrylate, polyquaternium-10, polyquaternium-11, polyquaternium-7, polyvinylpyrrolidone (PVP)/eicosene copolymer, PVP/hexadecene copolymer, and PVP/vinyl acetate (VA) copolymer.

The term “surfactant” means a compound which allows the surface tension of the composition to be reduced and promotes uniform distribution of the compounds in a cosmetic and/or dermatological composition. These compounds are known to those skilled in the art. For example, the surfactant may be chosen from the group comprising cetyl betaine, cocamidopropyl betaine, disodium cocoamphodiacetate, disodium laureth sulfosuccinate, hexylene glycol, laureth-3, laureth-4, polyethylene glycol (PEG) PEG-30, glyceryl cocoate, PEG-40 hydrogenated castor oil, PEG-7, glyceryl cocoate, polysorbate 20, polysorbate 80, potassium cetyl phosphate, sodium cetearyl sulfate, sodium laureth sulfate, sodium lauryl sulfate, sodium palmitate (sodium hexadecanoate), steareth-100, steareth-2 and steareth-21.

The term “moisturizer” means a compound which allows the water content of a cosmetic and/or dermatological composition to be increased. These compounds are known to those skilled in the art. For example, the moisturizer may be chosen from the group comprising aloe (Aloe vera), pineapple plant extract, dimethiconol, Echinacea angustifolia, glycine, licorice extract, jasmine extract, jojoba esters, magnesium stearate, mannitol, honey and castor oil.

The term “stabilizer” means a compound which makes it possible to improve the stability of a cosmetic and/or dermatological composition and/or makes it possible to improve the stability of the components included in said composition. These compounds are known to those skilled in the art. For example, the stabilizer may be chosen from the group comprising hydroxyethylcellulose, microcrystalline cellulose, polycaprolactone and sodium stannate.

The term “emulsion stabilizer” means a compound which makes it possible to improve the emulsification of a cosmetic and/or dermatological composition and/or to improve the stability of a cosmetic and/or dermatological emulsion. These compounds are known to those skilled in the art. For example, the emulsion stabilizer may be chosen from the group comprising acetylated glycol stearate, carbomer, cetearyl alcohol, Cyamopsis tetragonolubus, hydroxyethylcellulose, microcrystalline cellulose, PEG-90M, PVP/VA copolymer, sodium carboxymethyl dextran, stearic acid, octadecanol, synthetic paraffin wax and xanthan gum.

The term “absorbent agent” means a compound which absorbs dissolved or dispersed water-soluble and/or liposoluble substances. These compounds are known to those skilled in the art. For example, the absorbent agent may be chosen from the group comprising boron nitride, silicic acid, magnesium carbonate, microcrystalline cellulose, silica (silicon dioxide) and talc (E553).

The term “humectant” means any compound that is capable of maintaining and/or conserving the moisture content of a cosmetic and/or dermatological composition. These compounds are known to those skilled in the art. For example, the humectant may be chosen from butylene glycol, propylene glycol and polyethylene glycol.

The term “antioxidant” means any compound that is capable of inhibiting oxygen-mediated oxidation and rancidity reactions. These compounds are known to those skilled in the art. For example, the antioxidant may be chosen from the group comprising sulfites, amino acids, for example glycine, histidine, tyrosine, tryptophan and derivatives thereof, imidazoles, and peptides, for example D-carnosine or L-carnosine.

The term “fragrance” means a compound which enables a cosmetic and/or dermatological composition to be given a particular odor. These compounds are known to those skilled in the art. For example, the fragrance may be any fragrance that is suitable for cosmetic and/or dermatological application. The fragrance may be chosen, for example, from the group comprising natural plant extracts, plant essences, for example extracts and/or essences of roses, chamomile or mint.

Particularly preferably, the composition according to the invention may comprise at least one emollient, such as those mentioned previously (see point vi above). It may comprise between 10% and 60% by weight of emollient(s), depending on the type of formulation envisaged.

The composition according to the invention may also comprise one or more conventional complementary active agents, notably suitable for external topical administration, in particular dermatologically and cosmetologically acceptable active agents. These complementary active agents suitable for formulation are well known to those skilled in the art, and may be, for example, anti-aging active agents, soothing active agents, purifying active agents, moisturizing active agents, etc.

The composition may also comprise a means for facilitating its penetration into the skin. For example, a compound chosen from the group comprising vasodilators and glucocorticoids. This is of interest when the composition of the present invention is intended for topical application.

The cosmetic and/or dermatological composition of the invention may also comprise water. In this case, preferentially, the composition comprises demineralized or distilled water.

### Medical Device

The composition of the invention may also be administered via a medical device which comprises and discharges said composition in a controlled manner.

Any type of medical device allowing transdermal administration may be used to administer the composition according to the present invention. For example, the composition according to the present invention may be included in a gel applicable to the skin. The composition according to the present invention may be included in an adhesive sheet such as surgical-type ribbons, adhesive dressings, or even film dressings.

### Administration by Injection

In one embodiment according to the present invention, the invention may thus relate to the composition according to the invention for use by injection at a single site, or in a limited number of points in cutaneous or subcutaneous tissue. It may be repeated over time, notably at predefined time intervals, such as half-yearly.

In a particular embodiment, the invention may thus relate to the composition according to the invention for use in an intradermal injection.

According to a particular embodiment, the composition comprises at least one component suitable for administration by injection and at least one component suitable for external topical application, intended for application to the skin or its appendages.

The invention relates in particular to an injectable implant, in particular a dermal implant, administered via the subcutaneous or intradermal route. The invention thus relates to dermal implants, i.e. substances injected directly into the skin for the purpose of remedying disorders occurring at the various cutaneous or subcutaneous levels (dermis, hypodermis, etc.) described previously.

A compound or composition that is injectable, or suitable for administration by injection, is generally sterile. The composition is advantageously used as a combination product for simultaneous, separate or time-staggered use.

According to one embodiment, the component or composition suitable for administration by injection is in gel or implant form. These compositions may indeed be administered subcutaneously or intradermally.

In particular, they may be injectable implants or medical devices.

### The Systemic Route, More Particularly the Oral Route

One embodiment according to the present invention may thus relate to the composition according to the invention suitable for general (“systemic”) administration to an individual or an animal. Specifically, preliminary results (cf. examples) appear to show a certain activity of baicalin or a derivative thereof, more particularly in combination with chlorogenic acid or a derivative thereof, enabling systemic treatment to be envisaged.

The parenteral route may allow such administration (see above for more details in applicable embodiments).

The rectal or vaginal route may allow such administration. For example, a suppository formulation of the composition according to the invention is suitable for this purpose.

Preferably, the present invention relates to a composition that is suitable for oral administration.

Thus, a composition according to the present invention may take any form (solid or liquid) suitable for such oral administration, such as tablets, gel capsules, wafer capsules, powder, syrup, beverage, etc., optionally by means of a presentation form or device ensuring sustained and/or delayed release, such as a dissolution-retardant coating. For this type of formulation ensuring sustained and/or delayed release, an agent such as cellulose, carbonates or starch is advantageously used.

Another subject of the invention is a dietary supplement comprising a composition as described herein, which can be administered in the context of the cosmetic use or of the cosmetic treatment method of the invention. The formulations described above are suitable for such an application.

Thus, the invention also relates to an oral cosmetic treatment method for a composition according to the present invention.

The term “nutricosmetic” is an equivalent term which may be used when the composition according to the present invention is a dietary supplement.

Thus, a subject of the invention is also a cosmetic treatment method for improving the general appearance of the skin, comprising the oral administration of a composition according to the present invention.

The dietary supplement (or medicament) according to the present invention may be formulated for oral administration. It may thus be in wafer capsule, gel capsule, tablet or granule form, advantageously in wafer capsule form. When the dietary supplement or medicament is in soft capsule or gel capsule form, the envelope of these soft capsules or gel capsules may notably contain animal gelatin such as fish gelatin, glycerol, or a material of plant origin such as a cellulose or starch derivative, or a plant protein. When the dietary supplement or combination is in gel capsule, tablet or granule form, the active agent mixture may be fixed to a pulverulent support such as silica, cellulose or maltodextrin.

Thus, in a preferred manner, the composition according to the present invention is formulated to be a dietary supplement, for example in the form of tablets, gel capsules, wafer capsules, powders, syrups, beverages, etc., which may optionally be added to or dispersed in a conventional foodstuff.

In particular, the food supplement according to the present invention may advantageously comprise any suitable, nutraceutically acceptable vehicle or excipient, and also conventional additives, known to those skilled in the art. The dietary supplement and the combination can be used in combination with other active agents as a slimming action simultaneously, separately or staggered over time.

The food supplement may also be used simultaneously, separately or staggered over time with one or two disinfiltrating or venotonic active agents in addition to the combination or food supplement according to the invention. The disinfiltrating or venotonic active agents may be chosen from: viburnum, ivy, arnica, pisolla, wild pansy, Fucus vesiculosus, ruscus, Ginkgo biloba and escin.

### Applications

The cosmetic applications falling within the scope of the present invention may be chosen from the treatment or prevention of skin aging (wrinkles and fine lines), the cosmetic treatment or prevention of scarring or tattoo removal, the treatment or even prevention of skin coloration (improving the skin's structure improves blood passage, for example, which modifies the color of the skin which appears healthier).

Another subject of the invention is a use of the cosmetic and/or dermatological composition as described in the present invention, for combating the signs of skin aging.

In general, the amounts to be administered may be suitable for use according to the patient, the pathology, the disorder to be treated (i.e. cosmetically speaking), the mode of administration, etc. It is understood that repeated applications may be performed, optionally in combination with other active ingredients or any vehicle. In general, the daily dose of active agents falling within the context of the present invention (e.g. baicalin, chlorogenic acid) will be the minimum dose to obtain the desired dermatological and/or cosmetic effect.

In addition, the subject of the present invention relates to a composition comprising baicalin or a derivative thereof, more particularly in combination with chlorogenic acid or a derivative thereof, for use as a medicament.

Furthermore, for some of the compositions according to the present invention, activity on beta-defensin-2 release by human epidermal keratinocytes could be found.

More particularly, the subject of the present invention relates to a composition comprising baicalin or a derivative thereof, more particularly in combination with chlorogenic acid or a derivative thereof, for use in the cutaneous synthesis of hyaluronic acid and/or for use in an immunocutaneous response.

Specifically, improving the skin's structure (in particular in the case of systemic administration) allows the composition according to the present invention to be used therapeutically. For example, it allows the restoration from and/or prevention of cutaneous disorders, physiological or otherwise, including skin aging and impairment.

The composition can thus be used in human medicine, veterinary medicine, cosmetics and/or dermatology.

For example, said cosmetic composition may be used in order to prevent and/or treat skin disorders. These skin disorders may be of natural origin, pathological or linked to a therapeutic treatment. Disorders of natural origin are those linked to natural aging, for example skin aging.

For example, the invention relates to the composition according to the present invention for use in the treatment or prevention of a pathology caused by a microbial infection (fungal, bacterial and/or viral), a tumor or its consequences, skin aging (through the appearance of fine lines and wrinkles), hormonal skin aging, photo-induced aging, early skin aging, myo-lipo-cutaneous aging, lipodystrophy, restrictive dermopathy, dermatitis such as atopic dermatitis, psoriasis, acne, chemotherapeutic treatments, irradiation with X-rays, gamma rays, ultraviolet rays or, in general, consequences linked to side effects (impacting e.g. cellular synthesis of hyaluronic acid, or immunocutaneous response) of treatments.

The compositions according to the present invention may thus also comprise a fungicidal agent and/or an antibacterial agent, for example in the context of treating an immunocompromised patient or animal.

The term “fungicidal agent” (or equivalent term) means any compound that is capable of reducing and/or inhibiting the proliferation of fungi in a cosmetic and/or dermatological composition.

The term “antibacterial agent” (or equivalent term) means any compound that is capable of reducing or inhibiting the proliferation of bacteria in a cosmetic and/or dermatological composition. For example, the preserving agent may be chosen from the group comprising parabens, for example butylparaben, ethylparaben, propylparaben, isobutylparaben, methylparaben, organic acids, diazolidines, isothiazolinones, hydroxymethyl glycinates, phenolic alcohols, 5-bromo-5-nitro-1,3-dioxane, diazolidinylurea, 3′-demethoxy-30-demethylmatairesinol (DMDM) and hydantoin.

### Clauses

The present invention may be summarized on the basis of the clauses below: Clause 1: Cosmetic use of baicalin or a derivative thereof, optionally in combination with chlorogenic acid or a derivative thereof, in the cellular synthesis of hyaluronic acid, in particular in the skin.

Clause 2: Composition comprising baicalin or a derivative thereof, optionally in combination with chlorogenic acid or a derivative thereof, for use in the cellular synthesis of hyaluronic acid and/or in an immunocutaneous response.
Clause 3: Composition comprising baicalin or a derivative thereof in combination with chlorogenic acid or a derivative thereof.
Clause 4: Composition as claimed in clause 2 or 3, characterized in that, where appropriate, the ratio between the dry mass amount of baicalin or derivative thereof and the dry mass amount of chlorogenic acid or derivative thereof is between 1:1 and 1:10 respectively, preferentially between 1:3 and 1:5 respectively.
Clause 5: Composition as claimed in any one of clauses 2 to 4, characterized in that it is a preferentially aqueous liquid composition, or a solid such as a powder.
Clause 6: Composition as claimed in any one of clauses 2 to 5, characterized in that the concentration of baicalin or derivative thereof in the composition is less than 50 micromol, preferentially less than or equal to 25 micromol, of baicalin or derivative thereof per dm³ of composition and, where appropriate, in that the concentration of chlorogenic acid or derivative thereof in the composition is less than 200 micromol, preferentially less than or equal to 100 micromol, of chlorogenic acid or derivative thereof per dm³ of composition.
Clause 7: Composition as claimed in any one of clauses 2 to 6, characterized in that said composition comprises at least one of the compounds chosen from the following list: sodium hyaluronate, hydrolyzed sodium hyaluronate, sodium carboxymethyl beta-glucan, inulin, an alpha-glucan oligosaccharide, arginine and pentylene glycol.
Clause 8: Composition as claimed in any one of clauses 2 to 7, characterized in that said composition comprises between 0.1% and 5%, preferentially 2.00%, by mass of baicalin or a derivative thereof.
Clause 9: Composition as claimed in any one of clauses 2 to 8, characterized in that said composition comprises between 0.4% and 20%, preferentially 8.00%, by mass of chlorogenic acid or a derivative thereof.
Clause 10: Composition as claimed in any one of clauses 2 to 9, characterized in that said composition comprises between 0.8% and 40%, preferentially 16.00%, by mass of inulin.
Clause 11: Composition as claimed in any one of clauses 2 to 10, characterized in that said composition comprises between 0.2% and 10%, preferentially 4.00%, by mass of alpha-glucan oligosaccharide.
Clause 12: Composition as claimed in any one of clauses 2 to 11, characterized in that said composition comprises between 0.04% and 2.0%, preferentially 0.80%, by mass of sodium carboxymethyl beta-glucan.
Clause 13: Composition as claimed in any one of clauses 2 to 7, characterized in that said composition comprises:
baicalin or a derivative thereof, in particular between 0.0001% and 1% by mass of baicalin or a derivative thereof;
chlorogenic acid or a derivative thereof, in particular between 0.0004% and 4% by mass of chlorogenic acid or a derivative thereof; and/or
sodium carboxymethyl beta-glucan, in particular between 0.0004% and 0.8% by mass of sodium carboxymethyl beta-glucan.
   Clause 14: Composition as claimed in any one of clauses 2 to 7, characterized in that said composition comprises:
baicalin or a derivative thereof, in particular between 0.0001% and 1% by mass of baicalin or a derivative thereof;
chlorogenic acid or a derivative thereof, in particular between 0.0004% and 4% by mass of chlorogenic acid or a derivative thereof; and/or
inulin, in particular between 0.0008% and 8% by mass of inulin.
   Clause 15: Composition as claimed in any one of clauses 2 to 7, characterized in that said composition comprises:
baicalin or a derivative thereof, in particular between 0.0001% and 1% by mass of baicalin or a derivative thereof;
chlorogenic acid or a derivative thereof, in particular between 0.0004% and 4% by mass of chlorogenic acid or a derivative thereof; and/or
alpha-glucan oligosaccharide, in particular between 0.00002% and 2% by mass of alpha-glucan oligosaccharide.
   Clause 16: Composition as claimed in any one of clauses 2 to 10, characterized in that said composition comprises:
between 0.0001% and 1% by mass of baicalin or derivative thereof;
between 0.0004% and 4% by mass of chlorogenic acid or derivative thereof;
between 0.0004% and 0.8% by mass of sodium carboxymethyl beta-glucan;
between 0.0008% and 8% by mass of inulin; and/or
between 0.00002% and 2% by mass of alpha-glucan oligosaccharide.
   Clause 17: Composition as claimed in any one of clauses 3 to 16, for use as a medicament, or in the preparation of a medicament.
   Clause 18: Composition as claimed in any one of clauses 3 to 17, characterized in that the composition is a food supplement or a cosmetic composition, where appropriate.
   Clause 19: Composition as claimed in any one of clauses 7 to 19, for use in cellular synthesis of beta-defensin, in particular in cellular cutaneous synthesis of beta-defensin.
   Clause 20: Composition as claimed in any one of clauses 7 to 19, for use in the treatment of a disorder involving cellular synthesis of beta-defensin.
   Clause 21: Composition for use according to clause 20, characterized in that the disorder is a skin disorder chosen from microbial infection, restrictive dermopathy, dermatitis, such as atopic dermatitis, psoriasis and acne.

### FIGURES

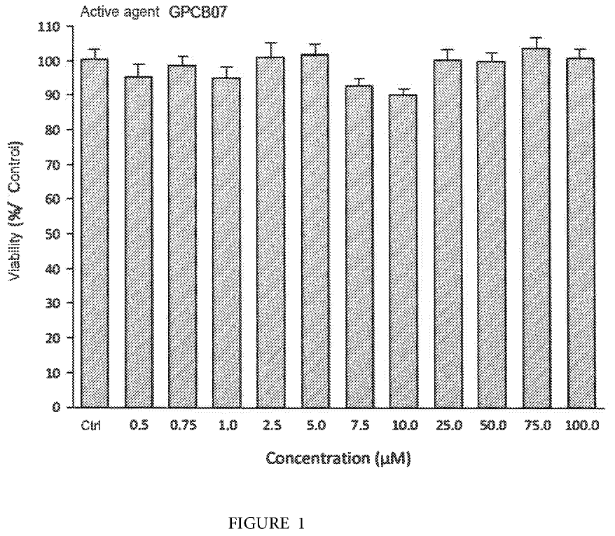

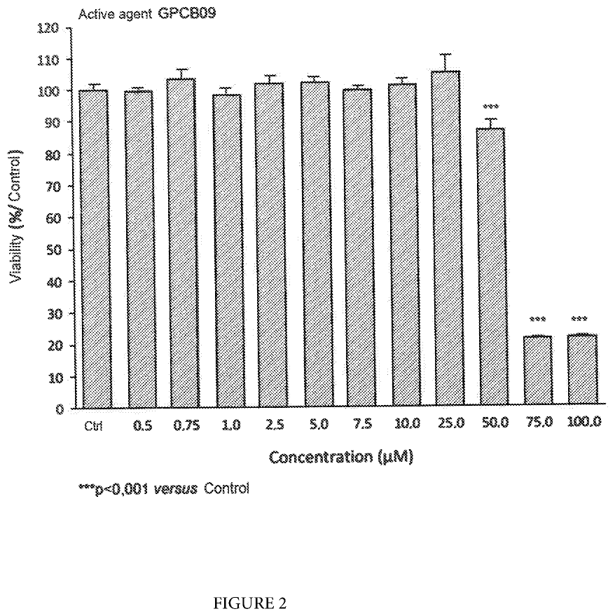

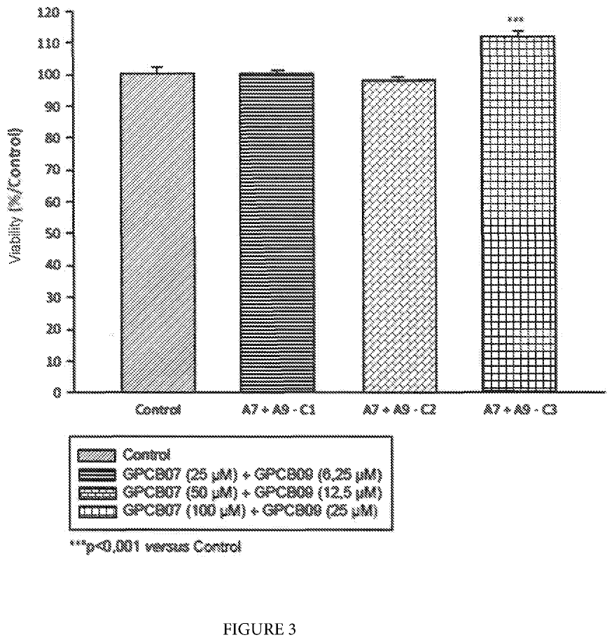

By way of nonlimiting examples, the figures found below correspond to:

[FIG. 1] represents a histogram showing cytotoxicity (cell viability) as a function of chlorogenic acid concentration;

[FIG. 2] represents a histogram showing cytotoxicity (cell viability) as a function of baicalin concentration;

[FIG. 3] represents a histogram showing cytotoxicity (cell viability) as a function of different mixtures of baicalin with chlorogenic acid.

[FIG. 4] represents a histogram showing the effect of a mixture of baicalin with chlorogenic acid (M41) and against chlorogenic acid (compound 7) and baicalin (compound 9) on cellular synthesis of hyaluronic acid.

### EXAMPLES

By way of nonlimiting examples, embodiments of the present invention are described below.

### A) Tests on Cellular Synthesis of Hyaluronic Acid

I. Materials and methods

### 1) Production and Culturing of Fibroblasts

Fibroblasts from healthy skin (FS) are cultured from biopsies performed on a 55-year-old female patient, on a skin explant derived from surgery and considered as surgical waste. The conventional explantation technique is used for fibroblast extraction. The explants are cultured in Dulbecco's modified Eagle's medium supplemented with 10% fetal calf serum (FCS), 40 mg/l gentamicin and 2 mg/l fungizone (DMEMc) at 37° C., in a humid atmosphere and in the presence of 5% CO2.

Gradually (over ten days), fibroblasts migrate outside the explants. The culture medium is changed twice a week. When sufficient amounts of fibroblasts are present, they are detached under the action of trypsin-EDTA, subcultured and amplified under the same culture conditions.

### 2) Cytotoxicity Study

### a) Principle

The study of cell viability in the presence of the active principles to be tested is performed by means of an MTT assay. MTT is a tetrazolium salt whose ring is transformed by mitochondrial dehydrogenases. The yellow substrate is transformed into a dark blue product by living cells, but not by dead cells or the culture medium. This colorimetric test allows the color intensity to be linked to the number of living cells. The absorbance at 550 nm is directly proportional to the number of living fibroblasts. Cytotoxicity occurs when the cell viability is less than 80% relative to the control.

### b) Preparation of Active Agent Stock Solutions

The active agents in powder form were taken up in 100% DMSO so as to obtain the stock solutions. Chlorogenic acid (GPCB07, molar mass=354.31 g/mol) and baicalin (GPCB09, molar mass=446.36 g/mol) were prepared at 100 μM. c) Procedure 96-well plates were seeded with 5000 fibroblasts per well. After 24 hours of culturing, the active principles to be tested were added. Twelve concentrations of each active principle were tested (n=10/concentration). The active agents were dissolved directly in the culture medium from the stock solutions. After 24 hours of culturing in the presence of the active agents, an MTT assay was performed.

Following the cytotoxicity results of the two active agents alone, a second cytotoxicity test was performed with the mixture. For each mixture, three concentrations were tested (n=10/concentration):
C1: highest concentration
C2: ½ dilution
C3: ¾ dilution

The mixture coded M41 corresponds to the mixture of products GPCB07 and GPCB09.

### 3) Fibroblast Culturing and Treatment

### a) Cell Culturing

At confluence, the fibroblasts were trypsinized and seeded in 6-well plates at 0.08×10⁶ cells per well in DMEMc 10% FCS. After 24 hours of culturing, the medium was replaced with DMEMc 1% FCS.

### b) Monolayer Treatment

After 24 hours of culturing, 2 ml of medium with or without active agent were added. The active agents were diluted to the concentrations chosen following cytotoxicity tests in DMEMc 1% FCS culture medium.

### c) Sampling

After 48 hours of culturing in the presence of the active agents, the supernatant was collected on antiprotease (10%). Aliquots were stored at −80° C. until assayed. The cell monolayers were washed with PBS and 500 μl of 0.1N NaOH were added. The cell lyzate was stored at −20° C. until protein assay.

### 4) Hyaluronic Acid Assay

### a) Principle

The amount of hyaluronic acid in the various samples was determined by ELISA (Enzyme-Linked Immunosorbent Assay) using a TECO@kit. This kit is a sensitive sandwich assay which uses microplates coated with hyaluronic acid binding protein (HABP) and HRP-conjugated HABP binding protein for detection. The HRP-conjugated HABP binding protein binds to the hyaluronic acid contained in the sample, and then reacts with the substrate. The color intensity is proportional to the HA content of the sample.

### b) Procedure

100 μl of standard or 100 μl of sample diluted to 1:50 were added to each well. The plate was incubated for 2 hours at room temperature, with agitation. After washing the plate, 100 μl of HRBP-HRP conjugate were added to each well. The plate was incubated for 30 minutes at room temperature, with agitation. After washing the wells, 100 μl of TMB substrate were added. The plate was incubated for 30 minutes at room temperature, in the dark and with agitation. The reaction was stopped by adding 100 μl of stop solution per well. Optical density readings were immediately taken at 450 nm.

### c) Calculations

The calibration curve was plotted, representing OD as a function of hyaluronic acid concentration. A regression curve was then plotted and the amounts of hyaluronic acid contained in the samples were determined by means of the equation of this regression curve. The hyaluronic acid concentrations are expressed in ng/mg of protein.

### 5) Protein Assay Using the Pierce Method

### a) Principle

Bicinchoninic acid (BCA), in sodium salt form soluble in aqueous medium, reacts stably, sensitively and highly specifically with cuprous ions. Proteins react with cupric ions in alkaline medium to produce cuprous ions. Two molecules of BCA react with one cuprous ion to form a violet complex which shows an absorption peak at 562 nm.

### b) Procedure

A calibration range (0-2 mg/ml) was established using a solution of bovine serum albumin. 20 μl of each point of the calibration or sample range were placed in duplicate in the wells of a 96-well plate. 200 μl of Pierce reagent were added and the plate was incubated for 30 minutes at 37° C. The absorbance was read immediately at 550 nm (MultiSkan FC spectrophotometer, Thermo).

### c) Calculations

The range allows a calibration curve to be plotted, from which the protein concentration of the samples is determined. The results are expressed in mg protein/ml.

### 6) Calculations and Statistical Analysis

The values are expressed as a percentage relative to the control, plus or minus the standard error of the distribution of the means (SEM). A one-factor analysis of variance is performed, followed if necessary by a Fisher test. Significance is retained only when p<0.05.

### II. Results

### 1) Cytotoxicity of Active Agents Alone

Active agent 7 (chlorogenic acid) had no effect on cell viability relative to the control, whatever the concentration.

Active agent 9 (baicalin) causes a significant reduction in cell viability at and above 50 μM relative to the control, notably for concentrations of 75 and 100 μM where the decrease is very significant.

TABLE 2
Chlorogenic acid

| Concentration (μM) | Viability (%/Ctrl) | SEM |
|---|---|---|
| Ctrl | 100 | 3.23 |
| 0.5 | 95.14 | 3.70 |
| 0.75 | 98.62 | 2.66 |
| 1.0 | 94.89 | 3.27 |
| 2.5 | 100.93 | 4.43 |
| 5.0 | 101.75 | 3.35 |
| 7.5 | 92.30 | 2.57 |
| 10.0 | 89.63 | 2.33 |
| 25.0 | 100.28 | 3.06 |
| 50.0 | 99.64 | 2.57 |
| 75.0 | 103.28 | 3.47 |
| 100.00 | 100.41 | 2.95 |

TABLE 3
Baicalin

| Concentration (μM) | Viability (%/Ctrl) | SEM |
|---|---|---|
| Ctrl | 100 | 1.80 |
| 0.5 | 99.82 | 1.09 |
| 0.75 | 103.65 | 2.93 |
| 1.0 | 98.27 | 2.35 |
| 2.5 | 101.73 | 2.50 |
| 5.0 | 101.92 | 1.84 |
| 7.5 | 99.55 | 1.39 |
| 10.0 | 100.76 | 2.24 |
| 25.0 | 104.77 | 5.54 |
| 50.0 | 86.65 | 3.05 |
| 75.0 | 21.41 | 0.19 |
| 100.00 | 21.80 | 0.18 |

The maximum non-cytotoxic concentrations retained for active agents 7 and 9 are 100 μM and 25 μM, respectively.

### 2) Cytotoxicity of the Active Agents as Mixtures

Mixture 41 significantly increased the cell viability at C3 relative to the control. There was no change in cell viability at C1 and C2 relative to the control.

TABLE 4
Chlorogenic acid (100 μM) and baicalin (25 μM) mixture

| | |
|---|---|
| Concentration (μM) | Viability % |

| Chlorogenic acid | Baicalin | Mean | SEM |
|---|---|---|---|
| 0 | 0 | 100.00 | 2.32 |
| 25 | 6.25 | 100.37 | 1.03 |
| 50 | 12.5 | 98.22 | 1.10 |
| 100 | 25 | 111.46 | 2.37 |

The maximum non-cytotoxic concentrations retained for the mixture are thus 100 μM and 25 μM for active agents 7 and 9, respectively.

### 3) Hyaluronic Acid Synthesis

We have shown that the M41 mixture consisting of 100 μM chlorogenic acid and 25 μM baicalin does not induce cytotoxicity in cells. This ratio was thus used to test the effect on hyaluronic acid synthesis. The effects of the products alone were also compared with those of the mixture.

Product 7 tested alone showed a slightly deleterious effect on hyaluronic acid synthesis, with a decrease of about 20% at the mean significance threshold.

Product 9 tested alone showed a slightly stimulating effect on hyaluronic acid synthesis, with an increase of about 30% at a low significance threshold.

On the other hand, the combination of the two products shows a highly significant synergistic effect on hyaluronic acid synthesis, with an increase of about 140%.

FIG. 4: Effect of the M41 mixture of compounds 7 and 9 on hyaluronic acid synthesis and comparison with the effect of the products separately.

Thus, significant synergism is observed at a concentration ratio of 4 in cell assays with the following concentrations: Compound 7: 0.004% and Compound 9: 0.001%

B) Effect on Antimicrobial Peptide Release from Keratinocytes

### I. Aim of the Study and Introduction

The study relates to an evaluation of the effect of four products/compositions on the release of beta-defensin-2 from normal human epidermal keratinocytes.

The products/compositions studied are as follows:
chlorogenic acid (“GPCB07”)
baicalin (“GPCB09”)
baicalin+chlorogenic acid (“GPCB07+GPCB09”)
aqueous composition of five active agents (called Premix; “GPCB11”) comprising baicalin (0.25% by mass), chlorogenic acid (1% by mass), sodium carboxymethyl beta-glucan (0.25% by mass) and a mixture of inulin and alpha-glucan oligosaccharide (2.5% by mass) with a mass ratio of inulin:alpha-glucan oligosaccharide of 8:2.

In the present study, the effect of the compounds GPCB07, GPCB09, GPCB07+GPCB09 and GPCB11 was investigated in normal human epidermal keratinocytes (NHEK) on the production of an antimicrobial peptide. More specifically, the effect of these compounds was evaluated on the release of beta-defensin-2 (hBD -2) by Enzyme-Linked Immunosorbent Assay (ELISA). Prior to this, a preliminary cytotoxicity test was performed using a standard WST-8 reduction assay to determine the concentrations to be tested.

### II. Materials and Methods

### 1) Biological Model

Cell type: Normal human epidermal keratinocytes (NHEK), used at the third passage
Culture conditions: 37° C., 5% CO2
Culture medium: Keratinocyte-SFM optimized for the test, supplemented with Epidermal Growth Factor and pituitary extract

### 2) Compounds Tested

TABLE 5

| | Appearance/ | Stock solution | Concentrations |
|---|---|---|---|
| Compounds tested | Storage | or intermediate | tested |
| GPCB07 | Powder | 10% (100 mg/ml) | 0.0033%, 0.01% |
| Batch No. 0397G1188 | Storage: RT | in DMSO | and 0.03% |
| MV211204-1 | | | |
| GPCB09 Batch No. | Powder | 8.9% (89 mg/ml) | 0.0001%, |
| 0260G0303 MV211204-2 | Storage: RT | in DMSO | 0.0003% and |
| | | | 0.001% |
| GPCB07 + GPCB09 | Powder | 10% (100 mg/ml) | 0.0003%, |
| Batch No. | Storage: RT | in DMSO | 0.001% and |
| 0397G1188/0260G0303 | | | 0.003% |
| MV21104-3 | | | |
| GPCB11 Batch No. | Liquid | 10% in culture | 0.0011%, |
| 070122 MV211204-4 | Storage: RT | medium | 0.0033% and |
| | | | 0.01% |

RT: Room temperature

### 3) Preliminary Test of Cytotoxicity

Cell type: NHEK in culture medium
Incubation time: 72 hours
Evaluation parameters: WST-8 reduction and morphological microscopic observations.

At the end of the treatment, the cells were incubated in the presence of WST-8 (a highly water-soluble tetrazolium salt), reduced to a water-soluble, orange-colored product (formazan) by succinate dehydrogenase (a mitochondrial enzyme). This transformation is proportional to the number of living cells and their metabolic activity. The optical density (OD) was measured with a spectrophotometer at 450 nm (VERSAmax, Molecular Devices).

### 4) Culturing and Treatment

The keratinocytes were seeded in 96-well plates and cultured in culture medium for 24 hours. The medium was then replaced with culture medium containing or not (control) the compounds to be tested or the reference cytokine mixture (IL-17+IL-22+TNF-α at 5 ng/ml each). The cells were then incubated for 72 hours. All the experimental conditions were performed in n=3.

### 5) ELISA Tests

TABLE 6

| | | Lower detection | Upper detection |
|---|---|---|---|
| ELISA kit | Supplier | limit (pg/ml) | limit (pg/ml) |
| “Human BD-2 | Peprotech, | 7.813 | 1000 |
| ABTS | Ref. 900-K172 | 39.065 | 5000 |
| Development” | Dilution factor | | |
| | 1/5 | | |

### 6) Data Processing

The inter-group comparisons were made using the unpaired bilateral Student's t-test. The statistical analyses can be interpreted if n>5; however for n<5, the calculated data are provided purely as a guide.

### Formulas Used:

Standard error of the mean: SEM=standard deviation (Sd)/fn

The standard error of the mean (SEM) represents the deviation of the sample mean relative to the true population mean. The SEM is calculated by dividing the Sd by the square root of the sample size.

Percentage viability: viability (%)=(OD compound/OD control)×100

### III. Results and Conclusion

### 1) Preliminary Test of Cytotoxicity

The results of the viability test using WST-8 and the observation of the cell lawns lead to the concentrations to be tested in the remainder of the study being selected (cf. Table 5).

TABLE 7
Effect of compounds GPCB07, GPCB09, GPCB07 + GPCB09
and GPCB11 on keratinocyte viability after 72-hour incubation.

| | | | GPCB07; Unit: mg/mL | | | | | | | |
|---|---|---|---|---|---|---|---|---|---|---|
| | | | Stock solution prepared at 100 mg/mL in DMSO | | | | | | | |
| GPCB07 | Control | | 0.00046 | 0.0014 | 0.0041 | 0.012 | 0.037 | 0.111 | 0.333 | 1.0 |
| Viability (%) | 101 | 103 | 104 | 104 | 103 | 99 | 96 | 90 | 77 | 18 |
| | 100 | 98 | 106 | 105 | 102 | 96 | 96 | 86 | 76 | 17 |
| | 98 | 100 | 102 | 102 | 101 | 96 | 95 | 85 | 75 | 16 |
| Mean (%) | 100 | | 104 | 104 | 102 | 97 | 96 | 87 | 76 | 17 |
| SEM (%) | 1 | | 1 | 1 | 1 | 1 | 0 | 1 | 1 | 1 |
| Morphological | + | | + | + | + | + | + | + | + | + |
| observations | | | | | | | | | | |

| | | | GPCB09; Unit: mg/mL | | | | | | | |
|---|---|---|---|---|---|---|---|---|---|---|
| | | | Stock solution prepared at 89 mg/mL in DMSO | | | | | | | |
| GPCB09 | Control | | 0.0004 | 0.0012 | 0.0037 | 0.011 | 0.033 | 0.098 | 0.287 | 0.380 |
| Viability (%) | 100 | 98 | 98 | 96 | 94 | 68 | 36 | 21 | 18 | 1 |
| | 100 | 99 | 100 | 94 | 81 | 65 | 34 | 20 | 18 | 2 |
| | 103 | 99 | 98 | 96 | 86 | 66 | 35 | 21 | 21 | 1 |
| Mean (%) | 100 | | 99 | 96 | 87 | 66 | 35 | 21 | 19 | 1 |
| SEM (%) | 1 | | 1 | 1 | 4 | 1 | 1 | 0 | 1 | 0 |
| Morphological | + | | + | + | + | + | + | +* | − | − |
| observations | | | | | | | | | | |

| | | | GPCB07 + GBC09; Unit: mg/mL | | | | | | | |
|---|---|---|---|---|---|---|---|---|---|---|
| GPCB07 + | | | Stock solution prepared at 100 mg/mL in DMSO | | | | | | | |
| GBC09 | Control | | 0.00046 | 0.0014 | 0.0041 | 0.012 | 0.037 | 0.111 | 0.333 | 1.0 |
| Viability (%) | 99 | 102 | 101 | 102 | 96 | 83 | 61 | 61 | 53 | 6 |
| | 97 | 100 | 100 | 101 | 95 | 81 | 61 | 59 | 51 | 7 |
| | 101 | 101 | 102 | 100 | 97 | 81 | 61 | 61 | 53 | 7 |
| Mean (%) | 100 | | 101 | 101 | 96 | 82 | 61 | 60 | 52 | 7 |
| SEM (%) | 1 | | 1 | 0 | 0 | 1 | 0 | 0 | 1 | 0 |
| Morphological | + | | + | + | + | + | + | +* | +* | +* |
| observations | | | | | | | | | | |

| | | | GPCB11; Unit: mg/mL | | | | | | | |
|---|---|---|---|---|---|---|---|---|---|---|
| | | | Stock solution prepared at 100 mg/mL in DMSO | | | | | | | |
| GPCB11 | Control | | 0.0046 | 0.014 | 0.041 | 0.123 | 0.370 | 1.1 | 3.3 | 10 |
| Viability (%) | 99 | 99 | 89 | 64 | 56 | 51 | 39 | 3 | 18 | 28 |
| | 101 | 100 | 89 | 65 | 57 | 52 | 41 | 4 | 17 | 26 |
| | 100 | 101 | 87 | 66 | 58 | 52 | 39 | 4 | 14 | 23 |
| Mean (%) | 100 | | 89 | 65 | 57 | 52 | 40 | 4 | 16 | 26 |
| SEM (%) | 0 | | 1 | 1 | 1 | 0 | 1 | 0 | 1 | 2 |
| Morphological | + | | + | + | +* | +* | +* | −* | −* | − |
| observations | | | | | | | | | | |

+: normal population
+/−: reduction in growth
−: toxicity
0: cell death
g: compound grains
op: opacity due to the compound
*Morphological changes
ag: agglutinated cells

2) Effect on hBD-2 Release

TABLE 8
Effect of compounds GPCB07, GPCB09, GPCB07 +
GPCB09 and GPCB11 on hBD-2 release from keratinocytes

| Compounds | | hBD-2 | Mean | SEM | % | SEM | |
|---|---|---|---|---|---|---|---|
| tested | Concentration | (pg/ml) | (pg/ml) | (pg/ml) | control | (%) | p^{(t)} |
| Controls | — | 13 | <11 | 2 | 100 | 15 | — |
| | | <8 | | | | | |
| | | 11 | | | | | |
| Mixture of | 5 ng/ml | 4306 | 4294 | 93 | 39979 | 863 | — |
| cytokines* | | 4128 | | | | | |
| (IL17 + | | 4449 | | | | | |
| IL22 + TNFα) | | | | | | | |
| GPCB07 | 0.0033% | <8 | <8 | 0 | <73 | 0 | ns |
| | | <8 | | | | | |
| | | <8 | | | | | |
| | 0.01% | <8 | <8 | 0 | <73 | 0 | ns |
| | | <8 | | | | | |
| | | <8 | | | | | |
| | 0.03% | <8 | <8 | 0 | <73 | 0 | ns |
| | | <8 | | | | | |
| | | <8 | | | | | |
| GPCB09 | 0.0001% | <8 | <8 | 0 | <73 | 0 | ns |
| | | <8 | | | | | |
| | | <8 | | | | | |
| | 0.0003% | <8 | <8 | 0 | <73 | 0 | ns |
| | | <8 | | | | | |
| | | <8 | | | | | |
| | 0.001% | <8 | <8 | 0 | <73 | 0 | ns |
| | | <8 | | | | | |
| | | <8 | | | | | |
| GPCB07 + | 0.0003% | <8 | <9 | 1 | <85 | 12 | ns |
| GPCB09 | | 12 | | | | | |
| | | <8 | | | | | |
| | 0.001% | <8 | <8 | 0 | <73 | 0 | ns |
| | | <8 | | | | | |
| | | <8 | | | | | |
| | 0.003% | <8 | <8 | 0 | <73 | 0 | ns |
| | | <8 | | | | | |
| | | <8 | | | | | |
| GPCB11 | 0.0011% | 14 | <10 | 2 | <90 | 18 | ns |
| | | <8 | | | | | |
| | | <8 | | | | | |
| | 0.0033% | <8 | <8 | 0 | <73 | 0 | ns |
| | | <8 | | | | | |
| | | <8 | | | | | |
| | 0.01% | 40 | 35 | 2 | 325 | 22 | ** |
| | | 32 | | | | | |
| | | 33 | | | | | |

p^{(t)}: p statistic for defining the significance threshold
*Statistical significance threshold
Ns: >0.05, not significant
*0.01 to 0.05, significant
** 0.001 to 0.01, very significant
*** <0.001, extremely significant
^{±} diluted sample
< or >: below or above the kit detection limit
<7.813 or >1000 pg/ml for diluted samples
<39.065 or >5000 pg/ml for ⅕ diluted samples
In the case where values are < or > detection limits, the calculations are extrapolated.

Under control conditions, the basal amount of hBD-2 released by normal human epidermal keratinocytes (“NHEK”) is negligible (<11 μg/ml) and, as expected, this release was very strongly stimulated by treatment with the combination of the three cytokines (IL-17+IL-22+TNF-α) (˜4300 μg/ml, i.e. -40 000% of control).

Baicalin and chlorogenic acid have no effect on beta-defensin-2 release from human epidermal keratinocytes. The combination of these two molecules also showed no effect on this target. The Premix had a significant effect on the release of beta-defensin-2 from human epidermal keratinocytes at a concentration of 0.01%.

### BIBLIOGRAPHICAL REFERENCES

REF 1—“The flavonoid baicalin exhibits anti-inflammatory activity by binding to chemokines”, Li B Q, Fu T, Gong W H, Dunlop N, Kung H, Yan Y, Kang J, Wang J M, Immunopharmacology 2000 September 49:295-306
REF 2—“Anti-inflammatory properties and inhibition of leukotriene c4 biosynthesis in vitro by flavonoid baicalein from Scutellaria baicalensis Georgi roots”, Butenko I G, Gladtchenko S V, Galushko S V. Agents Actions 1993; 39 Spec. No: C49-51
REF 3—“Pharmacological effects of methanolic extract of Scutellaria baicalensis root and its flavonoids on human gingival fibroblast”, Chung C P, Park J B, Bae K H, Planta Med. 1995 April 61:150-3 REF 4—“Scavenging effects of baicalin on free radicals and its protection on erythrocyte membrane from free radical injury”, Shi H, Zhao B, Xin W, Biochem. Mol. Biol. Int. 1995 April 35:981-94
REF 5—“Free radical scavenging and antioxidant activities of flavonoids extracted from the radix of Scutellaria baicalensis Georgi”, Gao Z, Huang K, Yang X, Xu H, Biochim. Biophys. Acta 1999 November 1472:643-50
REF 6—“Superoxide-scavenging and Tyrosinase-inhibitory Activities of the Extracts of Some Chinese Medicines”, Zhuang Miao, Hiroshi Kayahara, and Koji Tadasa, BBB Vol. 61 No. 12 1997
REF 7—“Effects of baicalein and alpha-tocopherol on lipid peroxidation, free radical scavenging activity and 12-O-tetradecanoylphorbol acetate-induced ear edema”, Hara H, Sukamoto T, Ohtaka H, Abe K, Tatumi Y, Saito Y, Suzuki A, and Tsukamoto G, Eur. J. Pharmacol. 1992 October 221:193-8
REF 8—“A comparative study of the anti-allergic effects of disodium baicalein 6-phosphate (BPS) and disodium cromoglycate (DSCG)”, Koda A, Watanabe S, Yanagihara Y, Nagai H, Sakamoto K, Jpn J. Pharmacol. 1977 Feb. 27:31-8
REF 9—“Inhibitory effects of certain natural products on hyaluronidase actuating and their anti-allergic actions”, Kakegawa H, Matsumoto H, Satoh T, Chem Pharm Bull (Tokyo) 1992 June 40:1439-42
REF 10—“Effects of luteolin and other flavonoids on IgE-mediated allergic reactions”, Kimata M, Inagaki N, Nagai H, Planta Med 2000 February 66:25-9
REF 11—“Inhibitory effect of baicalein, a flavonoid in scutellaria root, on eotaxin production by human dermal fibroblasts”, Nakajima T, Imanishi M, Yamamoto K, Cyong J C, Hirai K. Planta Med. 2001 March; 67(2):132-5.
REF 12—“The suppression of the N-nitrosating reaction by chlorogenic acid”, Kono Y, Shibata H, Kodama Y, Sawa Y, Biochem. J. 1995 December 312 (Pt 3):947-53
REF 13—“Structural damage to proteins caused by free radicals: evaluation, protection by antioxidants and influence of protein binding”, Aline Salvi, Pierre-Alain Carrupt, Jean-Paul Tillement and Bernard Testa, Biochemical Pharmacology, 2001, 61:10:1237-1242
REF 14—“Prevention of Skin Aging by Plant Extracts Acting on the Glycation Process Ex Vivo”, Bouzoud, D., Peno-Mazzarino, L., Arnold, F., Lati, E., Luu, M. T., Mercier, M. Poster presented at The Society of Cosmetic Chemists—Annual Scientific Meeting, New York, NY. (2010, December).
REF 15—“Effect of chlorogenic acid on melanogenesis of B16 melanoma cells”, Hao-Rong Li 1,2,3, Maidina Habasi 1,2, Lian-Zhen Xie 1,2,3 and Haji Akber Aisa Molecules 2014, 19, 12940-12948; doi:10.3390/molecules190912940
REF 16—“Effect of topical application of chlorogenic acid on excisional wound healing in rats”, Wei-Cheng Chen, Shorong-Shii Liou, Thing-Fong Tzeng, Shiow-Ling Lee, I-Min Liu, April 2013, Planta Medica 79(8) DOI: 10.1055/s-0032-132836
REF 17—“Effects of baicalein against UVA-induced photoaging in skin fibroblasts”, Wei Min, Xin Liu, Qihong Qian, Bingjiang Lin, Di-Wu, Miaomiao Wang, Israr Ahmad, Nabiha Yusuf, Dan Luo, (2014), Am. J. Chin. Med. 42, pages 709-727.
REF 18—“Cecropia obtusa extract and chlorogenic acid exhibit anti-aging effect in human fibroblasts and keratinocytes cells exposed to UV radiation”, Georgia de Assis Dias Alves, conceptualization, formal analysis, investigation, methodology, writing—original version, writing—revision and editing, Rebeca Oliveira de Souza, Methodology, Herv6 Louis Ghislain Rogez, Methodology, Writing revision and editing, Hitoshi Masaki, Supervision, and Maria Jos6 Vieira Fonseca, Conceptualization, Formal analysis, Funding acquisition, Supervision, Writing -revision and editing, Partha Mukhopadhyay, Editor, published online May 8, 2019, doi: 101371/journal.pone.0216501.
REF 19—“Tracking Skin and Immune Cell Interactions”, Chae Ho Lim, Mayumi Ito, Nature Cell Biology, 23, 439-440 (2021)

1. (canceled)

2. A composition comprising baicalin or a derivative thereof, optionally in combination with chlorogenic acid or a derivative thereof, for cellular synthesis of hyaluronic acid and/or immunocutaneous response.

3. A composition comprising:
baicalin or a derivative thereof in combination with chlorogenic acid or a derivative thereof.

4. The composition as claimed in claim 2, wherein the ratio between the dry mass amount of baicalin or derivative thereof and the dry mass amount of chlorogenic acid or derivative thereof is between 1:1 and 1:10 respectively.

5. The composition as claimed in claim 2, wherein said composition is an aqueous liquid composition, or a solid powder.

6. The composition as claimed in claim 2, wherein the concentration of baicalin or derivative thereof in the composition is less than 50 micromol, of baicalin or derivative thereof per dm³ of composition and, in that the concentration of chlorogenic acid or derivative thereof in the composition is less than 200 micromol of chlorogenic acid or derivative thereof per dm³ of composition.

7. The composition as claimed in claim 2, wherein said composition comprises at least one of the compounds chosen from the following list: sodium hyaluronate, hydrolyzed sodium hyaluronate, sodium carboxymethyl beta-glucan, inulin, an alpha-glucan oligosaccharide, arginine and pentylene glycol.

8. The composition as claimed in claim 2, wherein said composition comprises:
between 0.0001% and 1% by mass of baicalin or derivative thereof;
between 0.0004% and 4% by mass of chlorogenic acid or derivative thereof,
between 0.0004% and 0.8% by mass of sodium carboxymethyl beta-glucan;
between 0.0008% and 8% by mass of inulin; and/or
between 0.00002% and 2% by mass of alpha-glucan oligosaccharide.

9. The composition as claimed in claim 3, wherein the composition is a medicament.

10. The composition as claimed in claim 3, wherein the composition is a food supplement or a cosmetic composition, where appropriate.

11. The composition as claimed in claim 3, wherein the ratio between the dry mass amount of baicalin or derivative thereof and the dry mass amount of chlorogenic acid or derivative thereof is between 1:1 and 1:10 respectively.

12. The composition as claimed in claim 2, wherein the ratio between the dry mass amount of baicalin or derivative thereof and the dry mass amount of chlorogenic acid or derivative thereof is between 1:3 and 1:5 respectively.

13. The composition as claimed in claim 11, wherein the ratio between the dry mass amount of baicalin or derivative thereof and the dry mass amount of chlorogenic acid or derivative thereof is between 1:3 and 1:5 respectively.

14. The composition as claimed in claim 3, wherein said composition is an aqueous liquid composition, or a solid powder.

15. The composition as claimed in claim 2, wherein the concentration of baicalin or derivative thereof in the composition is less than 25 micromol, of baicalin or derivative thereof per dm³ of composition and, in that the concentration of chlorogenic acid or derivative thereof in the composition is less than 100 micromol of chlorogenic acid or derivative thereof per dm³ of composition.

16. The composition as claimed in claim 3, wherein the concentration of baicalin or derivative thereof in the composition is less than 50 micromol of baicalin or derivative thereof per dm³ of composition and in that the concentration of chlorogenic acid or derivative thereof in the composition is less than 200 micromol of chlorogenic acid or derivative thereof per dm³ of composition.

17. The composition as claimed in claim 16, wherein the concentration of baicalin or derivative thereof in the composition is less than 25 micromol of baicalin or derivative thereof per dm³ of composition and in that the concentration of chlorogenic acid or derivative thereof in the composition is less than 100 micromol of chlorogenic acid or derivative thereof per dm³ of composition.

18. The composition as claimed in claim 3, wherein said composition comprises at least one of the compounds chosen from the following list: sodium hyaluronate, hydrolyzed sodium hyaluronate, sodium carboxymethyl beta-glucan, inulin, an alpha-glucan oligosaccharide, arginine and pentylene glycol.

19. The composition as claimed in claim 3, wherein said composition comprises:
between 0.0001% and 1% by mass of baicalin or derivative thereof;
between 0.0004% and 4% by mass of chlorogenic acid or derivative thereof,
between 0.0004% and 0.8% by mass of sodium carboxymethyl beta-glucan;
between 0.0008% and 8% by mass of inulin; and/or
between 0.00002% and 2% by mass of alpha-glucan oligosaccharide.
